(19) United States
(12) Patent Application Publication
(10) Pub. No.: US 2025/0281496 A1
(43) Pub. Date: Sep. 11, 2025

(54) METHODS OF TREATING NEUROPATHY USING FORETINIB AND COMPOSITIONS THEREOF

(71) Applicant: INDIANA UNIVERSITY RESEARCH AND TECHNOLOGY CORPORATION, Bloomington, IN (US)

(72) Inventors: Konstantin FEINBERG, Indianapolis, IN (US); Gregory H. BORSCHEL, Indianapolis, IN (US)

(21) Appl. No.: 18/861,393

(22) PCT Filed: May 1, 2023
(86) PCT No.: PCT/US2023/020610

§ 371 Date: Oct. 29, 2024

Related U.S. Application Data
(60) Provisional application No. 63/336,669, filed on Apr. 29, 2022.

(51) Int. Cl. A61K 31/5377; A61K 9/00; A61P 25/02; A61P 25/28

(52) U.S. Cl. CPC A61K 31/5377; A61K 9/0019; A61P 25/02; A61P 25/28

(57) ABSTRACT

The present invention provides methods, formulations, and compositions for treating neurodegeneration in a subject.

### CROSS-REFERENCE TO RELATED APPLICATIONS

The present application claims priority to U.S. Provisional Patent Application No. 63/336,669 that was filed Apr. 29, 2022, the entire contents of which are hereby incorporated by reference.

### BACKGROUND

To date, 1 in 11 adults, or 463 million people worldwide, are estimated to have diabetes, a number that has more than tripled over the past two decades¹. As a result, approximately 10% of all global health expenditures (760 billion USD) are spent on diabetes¹. Of those 463 million diabetics worldwide, up to 50% eventually develop a neurodegenerative disease termed diabetic neuropathy² with loss of sensation, paresthesia, and, in one-third of patients, persistent pain³⁻⁵. This makes diabetes the most common cause of neuropathy to date⁶. The most common form is the distal symmetric neuropathy⁷, usually affecting the distal extremities and primarily the lower limbs and feet in a stocking pattern. These patients develop impaired foot sensation and commonly develop debilitating foot ulcers that are expensive and difficult to treat^{8,9}.

In diabetic neuropathy, neurodegeneration occurs in a length dependent fashion and primarily in sensory and autonomic axons of the peripheral nervous system¹⁰. Whilst the neuronal cell bodies are initially preserved, the axon terminals in the periphery, such as intraepidermal nerve fibers, are affected first, preceding the axonal loss in the proximal limb³. However, diabetes targets the entire neuron as cell bodies alter their phenotype in chronic diabetes, thereby likely contributing to a lack of structural support for distal axon branches¹¹. Further, in advanced diabetic neuropathy, Schwann cells are affected by chronic hyperglycemia which can lead to nerve fiber demyelination and/or Schwann cell dysfunction, potentially aggravating the sensory and autonomic symptoms^{12,13}

Although the pathomechanism of this characteristic pattern of neurodegeneration is subject to an ongoing debate, recent work indicated that hyperglycemia-induced nutrient excess in neurons causes phenotypic alterations in mitochondrial biologyl⁴. The altered activity of respiratory chain components in mitochondria in diabetic neurons results in reduced adaptability to fluctuating energy demand and thus may cause exhaustion of the ATP supply in distal axonal components¹⁴. The consequences of insufficient ATP supply in axon terminals may include gradual cytoskeletal breakdown and axonal pruning. In conjunction with an impaired ability for collateral sprouting and axon regeneration this contributes to the progressive denervation of sensory and autonomic distal targets^{14,15}

Despite the massive consequences of diabetic neuropathy, current treatment strategies fail to prevent or reverse axonal loss in diabetic patients. Thus, there is a need for new therapies for neuropathy.

### SUMMARY

In an aspect of the disclosure, methods of treating neurodegeneration in a subject in need thereof are provided. In some embodiments, the methods comprise administering a therapeutically effective amount of foretinib to the subject to treat the neurodegeneration.

In another aspect of the current disclosure, methods of restoring motor control in a subject with amyotrophic lateral sclerosis (ALS) are provided. In some embodiments, the methods comprise administering a therapeutically effective amount of foretinib to the subject to restore motor control in the subject.

In another aspect of the current disclosure, methods of reducing myelin loss in peripheral nerves in a subject suffering from multiple sclerosis are provided. In some embodiments, the methods comprise administering a therapeutically effective amount of foretinib to the subject to reduce myelin loss in peripheral nerves.

In another aspect of the current disclosure, methods of improving cutaneous wound healing associated with diabetic neuropathy in a subject in need thereof. In some embodiments, the methods comprise administering a therapeutically effective amount of foretinib to the subject to improve cutaneous wound healing in the subject.

In another aspect of the current disclosure, methods of increasing the axonal length of a neuron are provided. In some embodiments, the method comprising contacting the neuron with an effective amount of foretinib to increase the axonal length of the neuron.

In another aspect of the current disclosure, methods of increasing axonal length of a neuron in a subject are provided. In some embodiments, the methods comprise administering a therapeutically effective amount of foretinib to the subject to increase the axonal length of the neuron in the subject.

In another aspect of the current disclosure, pharmaceutical compositions are provided. In some embodiments, the pharmaceutical compositions comprise foretinib and one or more pharmaceutically acceptable carrier or excipients, wherein the pharmaceutical composition is formulated for ophthalmic topical use.

In another aspect of the current disclosure, methods of increasing innervation of the cornea in a subject in need thereof are provided. In some embodiments, the methods comprise administering a therapeutically effective amount of foretinib to the subject to increase corneal innervation in the subject.

### BRIEF DESCRIPTION OF THE DRAWINGS

FIG. 1 shows the diabetic phenotype in experimental animals. The dashed line represents the diabetic threshold of 15 mmol/l. Diabetic groups had significantly higher glucose compared to the non-diabetic control. Between diabetic groups there were no significant differences except for week 4 when the Foretinib treated group had significantly lower blood sugar values compared to the vehicle treated control (p=0.0036; mean±SEM; * indicates significant differences at p<0.05).

FIGS. 2A-2I show the cutaneous innervation in experimental and control animals. (A) Fluorescent image of a longitudinally cross sectioned plantar full-thickness skin sample in maximum intensity projection of a non-diabetic control showing a densely innervated epidermis. The nuclear counterstaining in blue (DAPI) and nerve fibers in green (beta 3). The red dotted line delineates the dermo-epidermal junction and red arrows indicate intraepidermal nerve fibers. (B) Plantar full-thickness skin sample of a diabetic mouse treated with a vehicle, showing a reduced IENFD. (C) Plantar full-thickness skin sample of a diabetic Foretinib treated mouse, showing a similarly reduced IENFD. Scale bars represent 15 μm. (D) Representative maximum intensity projection image of a plantar full-thickness skin sample of a non-diabetic control showing a dense cutaneous nerve fiber plexus in green (thy-1 yfp). (E) Plantar full-thickness skin sample of a non-treated diabetic mouse with a loss of smaller, interconnecting dermal nerve fiber bundles but remaining larger nerve fiber trunks in the deep dermis and subcutis. (F) Plantar full-thickness skin sample of a Foretinib-treated diabetic mouse showing a largely preserved but subepidermal nerve fiber plexus compared to D. (G) Plantar full-thickness skin sample of a vehicle-treated diabetic mouse showing the marked reduction of dermal nerve fiber density, comparable to C. Scale bars indicate 100 μm. (H) Plantar intraepidermal nerve fiber density (IENDF) of experimental animals 5 weeks post-diabetes induction showing that all diabetic groups had a significantly reduced IENDF compared to the healthy control (p<0.01), with no significant difference between diabetic groups. (I) Cutaneous nerve fiber density in a full thickness plantar skin sample indicating a neuroprotective effect of Foretinib on cutaneous nerve fibers in diabetic mice. * Indicates significant differences (p<0.05); **(p<0.01); ***(p<0.001); **** (p<0.0001); Epi=epidermis.

FIGS. 3A-3P show the histomorphology of lower extremity nerves in experimental and control animals. (A) Drawing that illustrates the site of neuro-histomorphologic analysis of the lower extremity nerves in mice. The sciatic nerve was analyzed approximately 5 mm proximal to its bifurcation and the sural nerve was analyzed at the ankle level. (B) Representative cross section of a sciatic nerve from a non-diabetic mouse, containing separate fascicles. The red dashed square indicates the position of the magnified image in C. Scale bar 100 μm. (C) Sciatic nerve of a non-diabetic mouse in higher magnification. (D) Sciatic nerve of a diabetic mouse. (E) Sciatic nerve of a Foretinib-treated diabetic mouse. (F) Sciatic nerve of a vehicle-treated diabetic mouse. (G) Axon diameter of the sciatic nerve. (H) Myelin sheath thickness of the sciatic nerve. (I) G-ratio, as axon diameter divided by total nerve fiber diameter, of the sciatic nerve. No significant differences between groups for all metrics (p>0.05). (J) Axon diameter of the sural nerve at the ankle level. (K) Myelin sheath thickness of the sural nerve. (L) G-ratio of the sural nerve. No significant differences between groups for all metrics (p>0.05). (M) Sural nerve cross section of a non-diabetic mouse. (N) Sural nerve of a diabetic mouse. (O) Sural nerve of a Foretinib-treated diabetic mouse. (P) Sural nerve of a vehicle-treated diabetic mouse. Scale bar 10 μm. Osmium stained, epoxy embedded nerves, 1 μm cross sections.

FIGS. 4A-4B show potential side effects of Foretinib. (A) Body weight of the experimental animals with the vehicle treated group being significantly lighter compared to the non-treated diabetic group in week 2 and 4 (p<0.05). (B) Relative body weight as proportion of the pre-diabetic weight showing that the Foretinib treated group dropped below the threshold of −15% weight loss at 5 weeks post-injection (dashed line). The Graphs display mean±SEM. * indicates significant differences (p<0.05).

FIG. 5 shows that Foretinib supports myelination. A) Embryonic rat sensory neurons were harvested, dissociated and cultured. Myelination was induced in presence of Foretinib (right image) or vehicle only (left image). 10 days since induction of myelination the cultures were fixed and immunosatined for myelin basic protein (MBP) (green), bIII-tubulin (red) and DAPI (blue). Scale bar: 500 mm. B) Quantitative representation of the area proportion occupied by MBP-positive myelin segments (as in A) n=4, p<0.0001.

FIGS. 6A-6E show that Foretinib induces axonal growth in pathological conditions. A) Rat sympathetic neurons grown in compartmented cultures for one week in the presence of NGF were switched into the indicated conditions for 48 h and immunostained for βIII-tubulin (white). Schematics on the left show the configurations used. In the top panels, NGF was maintained in all compartments. In the center and bottom panels, NGF was withdrawn from all compartments. At the bottom panel and 500 nM foretinib (Foret) added to one of the sides and the center compartment. Fluorescence images show βIII-tubulin-positive axons in the sides compartment. At the right, enlarged images of the axons per condition. While the NGF deprived neurons degenerated (axonal beading and degradation in the central panel), Foretinib supplemented (lower panel) axons kept growing Red arrow at the top of the image indicates the distance of the axonal extension during 48 h since NGF withdrawal. Size bar: 500 mm.B) MNs were differentiated from healthy human iPS (Line ID 1016A)⁶² as described⁶³. 3 day old MNs were treated with either 1 μM of Thapsigargin (Thaps) or 1 μM Tunicamycin (Tuni) and/or with either 0.5 uM of Foretinib. 48 hrs after treatment, the cells were fixed and immunostained for Islet1/2, bIII-tubulin (TUJ1) and labeled with Hoechst. C) Quantitative representation of the results as in B. Error bars represent standard error; *p<0.05; ***p<0.005; t-test. D) GFP-expressing MN in 2 days old TDP-43 mutant Zebra fish larvae (Hb9:Tg:tardbp)⁴⁶. MN axons of the untreated, mutant TDP-43 fish (left) are shorter and branched with extending diagonally neurites (arrowheads). The MN of larva exposed to Foretinib (1 mM) are longer and forms more NMJ (right, arrows), comparable to a wt larvae⁶⁴. E) Quantitative representation of changes in mouse body weight in dependence of the indicated IP daily doses of Foretinib.

FIGS. 7A-7B shows the response of mice to varied daily dosages and IP injected foretinib and the pharmacokinetics of the varied dosages. A) Quantitative representation of changes in mouse body weight in dependence of the indicated IP daily doses of Foretinib (n=2 per dose). B) Changes in mouse blood plasma concentration of 5 mg/kg IP injected Foretinib during 48 hours after a single injection (n=3).

### DETAILED DESCRIPTION

Disclosed are methods, formulations, and compositions for treating neurodegeneration in a subject. As used herein “neurodegeneration” refers any pathological condition in which the a neuron loses its function, structure, or both. Neurodegeneration may lead to neuropathy in a subject, as the declining, or lack of, function of neurons results in abberant function of the nervous system.

Neuropathic pain (or neuropathy) is caused by disorders of the nervous system. Neuropathic pain is typically accompanied by tissue damage, including nerve fibers that are damaged, dysfunction or injured, e.g., by neurodegeneration. Neuropathic pain may be caused by a variety of problems, including pathologic lesions, neurodegeneration processes, or prolonged dysfunction of parts of the peripheral or central nervous system. Neuropathic pain can also be present when no detectable damage can be assessed or defined.

Neuropathic pain as having two components: central plasticity and changes in peripheral nerves. Central plasticity can be the result of changes in receptor population or receptor sensitivity at any level of the CNS, or changes taking place in neurons and in microglia. Microglial activity is an important mediator of central sensitization of the spinal cord. Such central sensitization is known to play a major role in mediating chronic inflammatory as well as neuropathic pain.

In the periphery, changes in interaction between Schwann cells and axons play a role in the induction and maintenance of neuropathic pain.

### Methods of Treating Neuropathy

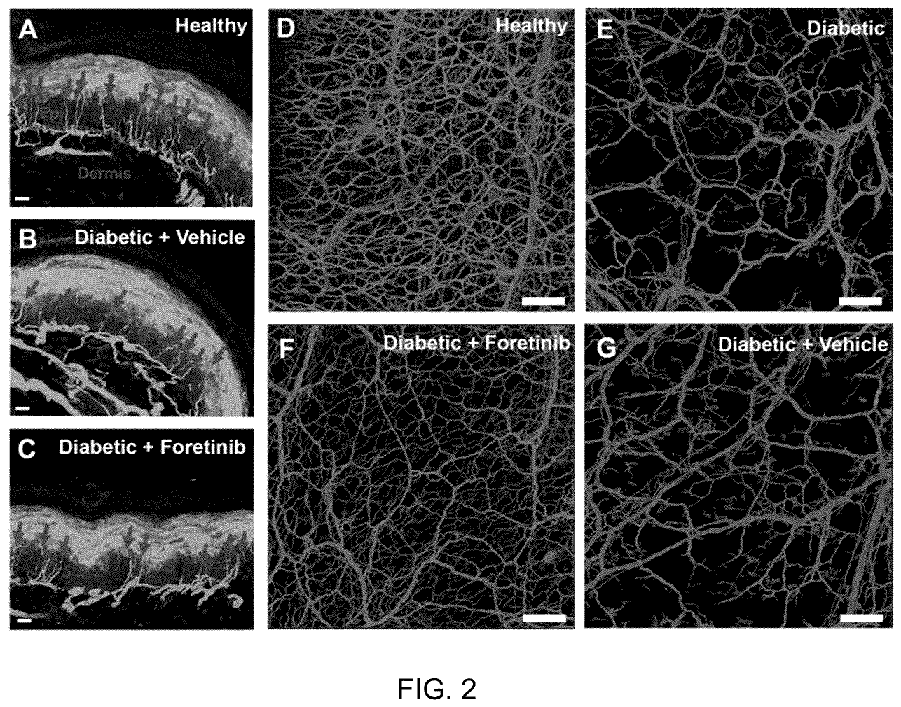

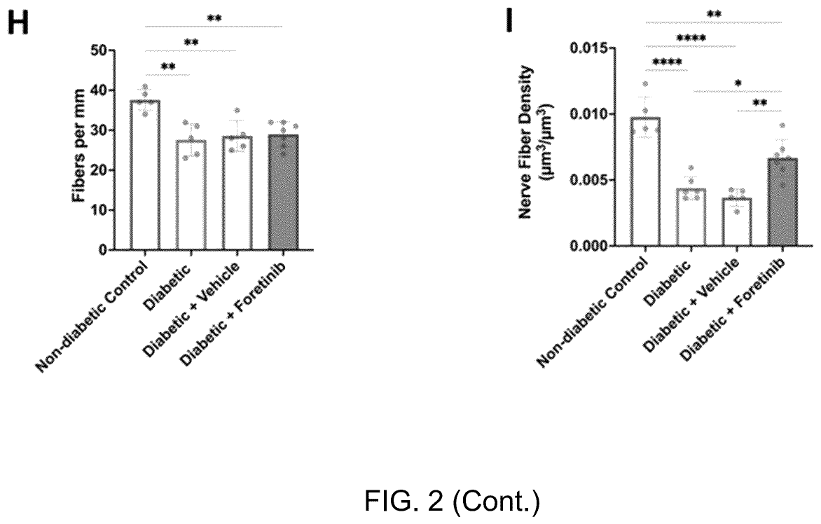

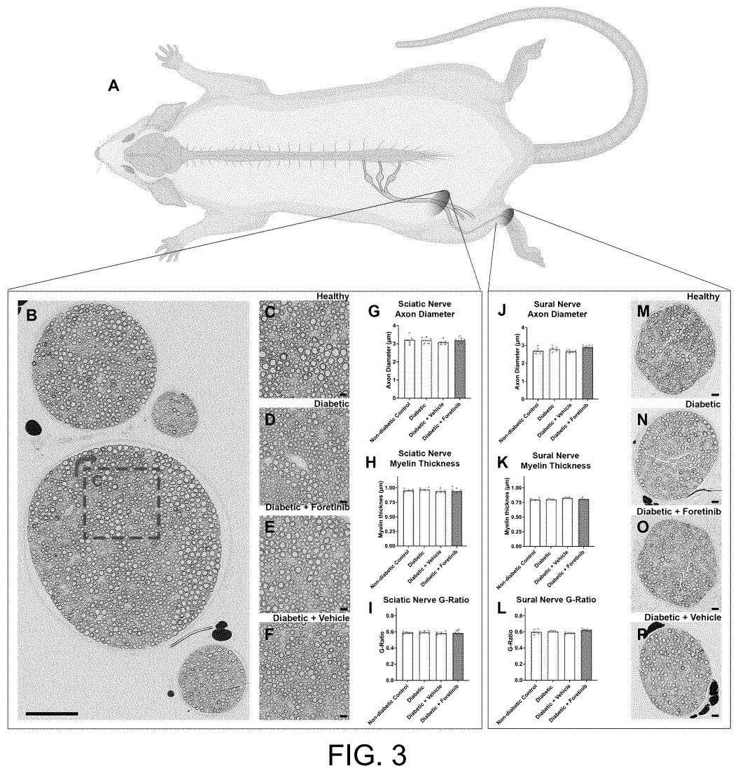

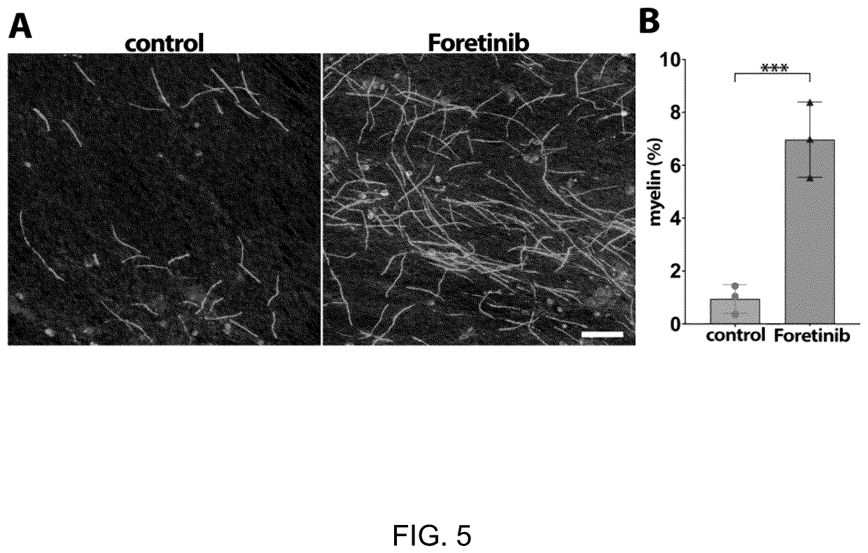

In a first aspect, the instant disclosure provides methods of treating neuropathy in a subject in need thereof. In some embodiments, the methods comprise administering an effective amount of foretinib to the subject to treat the neuropathy. Treatment results in the reduction in one or more symptom associated with neuropathy. The inventors have surprisingly found that the treatment with foretinib, while resulting in a lack of apparent effect on the proximal nerve morphology, demonstrates a surprising improved intradermal innervation in diabetic animals with neuropathy (FIGS. 2 and 3). Foretinib is the only compound that the inventors tested that demonstrated such an effect and may likely provide an improved and superior treatment option for neuropathy. Examples of neuropathies that may be treated with foretinib include diabetic neuropathy, multiple sclerosis (MS), and amyotrophic lateral sclerosis (ALS). Furthermore, the inventors found that, surprisingly, foretinib increased the density of myelination in cultured peripheral nerves (FIG. 5).

As used herein, neuropathy refers to damage to nerves. In some embodiments, neuropathy is “diabetic neuropathy.” Diabetic neuropathy refers to neuropathy secondary to diabetes. In some embodiments, the neuropathy is MS or ALS. The subject may have end-stage ALS. The methods may induce the growth of new neuromuscular junctions in a subject. The method may improve respiration, swallowing, or both respiration and swallowing in a subject.

As used herein, a “subject” may be any mammal, suitably a human, domesticated animal such as a dog, cat, horse, cow, pig, or a mouse or rat. A “subject in need thereof” is, in some embodiments, a subject diagnosed with a neuropathy, or a subject suffering from neuropathy, or a subject suspected of developing neuropathy in the future. In some embodiments, a subject in need thereof is a subject that has been diagnosed with a neuropathy, such as diabetic neuropathy, ALS, or MS, or that has been diagnosed with diabetes, or who has chronically elevated blood sugar who is at risk of developing diabetic neuropathy, or who has chronically elevated blood sugar who is at risk of developing diabetic neuropathy. In some embodiments, a subject in need thereof has been diagnosed with type 1 diabetes. The method may not reduce blood glucose levels in the subject. In some embodiments, a subject in need thereof has been diagnosed with type 2 diabetes. A subject in need thereof may be a subject diagnosed with multiple sclerosis (MS) or amyotrophic lateral sclerosis (ALS).

Diabetic neuropathy may be caused by prolonged elevated blood glucose or lipids due to diabetes. There are different types of neuropathy: peripheral neuropathy, autonomic neuropathy, focal neuropathy, and proximal neuropathy. Peripheral neuropathy is nerve damage that typically affects the feet and legs and sometimes affects the hands and arms. Autonomic neuropathy is damage to nerves that control the internal organs. Autonomic neuropathy can lead to problems with heart rate and blood pressure, digestive system, bladder, sex organs, sweat glands, eyes, and ability to sense hypoglycemia. Focal neuropathies are conditions in which one typically has damage to single nerves, most often in the hand, head, torso, and leg. Proximal neuropathy is a rare and disabling type of nerve damage in the hip, buttock, or thigh. This type of nerve damage typically affects one side of the body and may rarely spread to the other side. Proximal neuropathy often causes severe pain and may lead to significant weight loss.

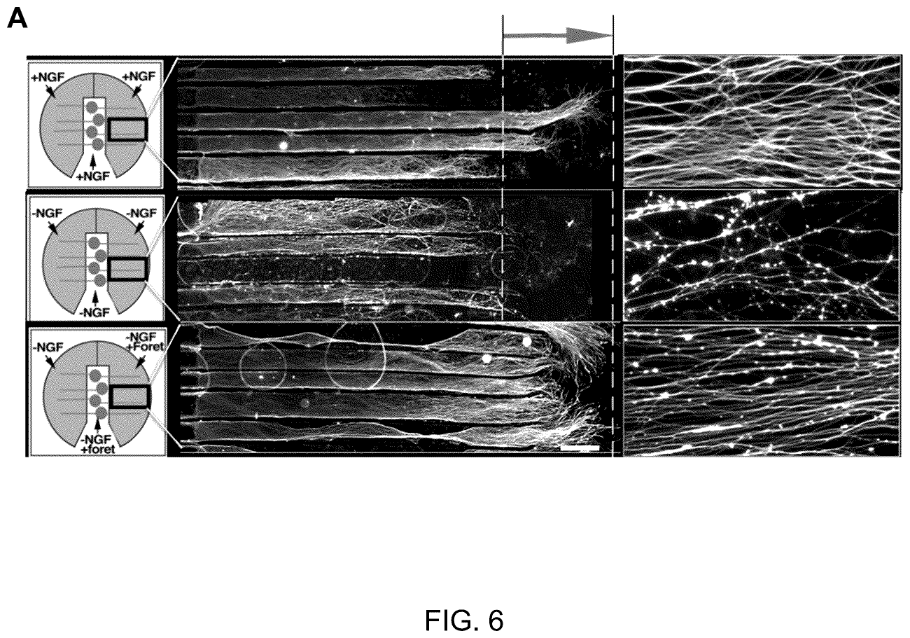

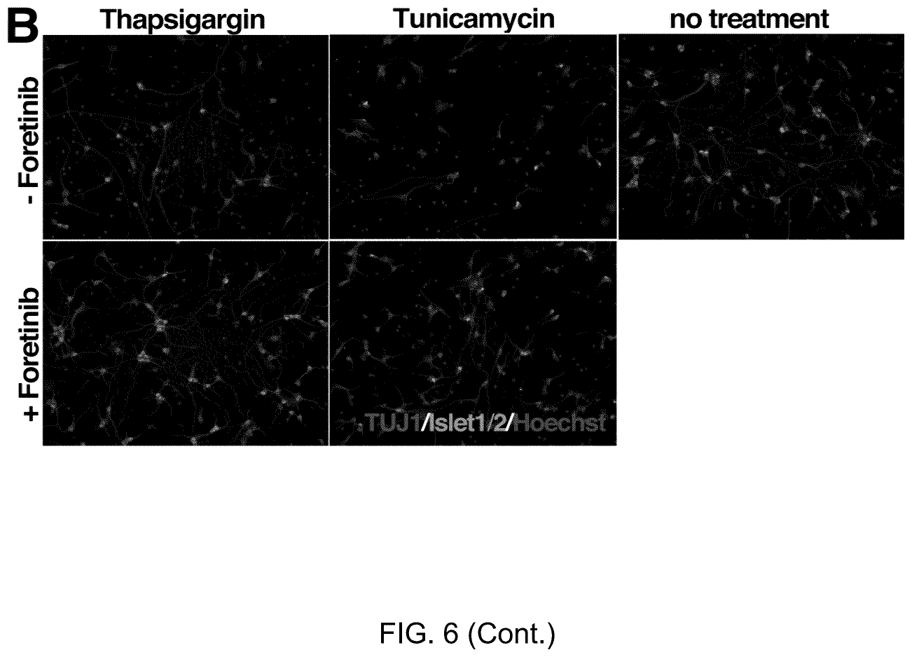

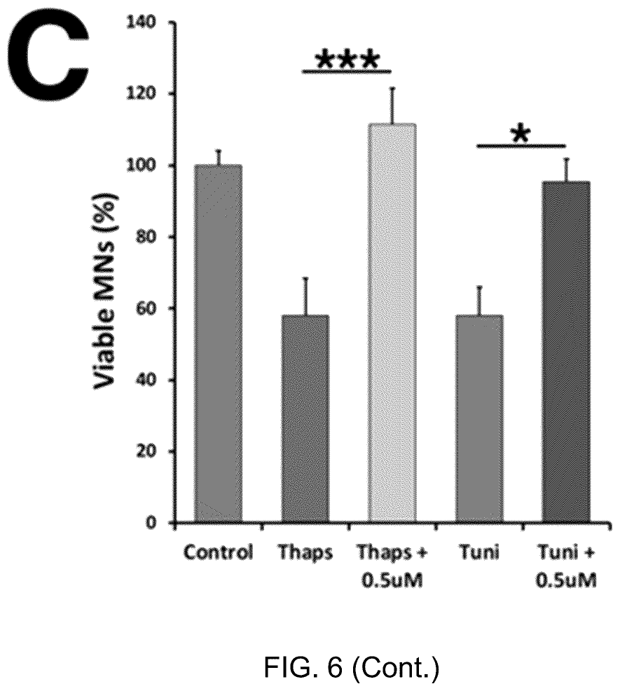

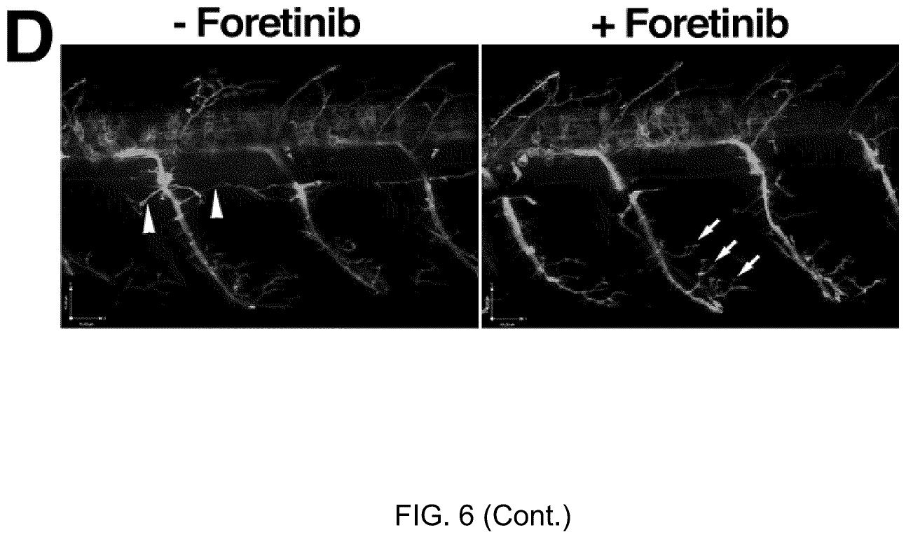

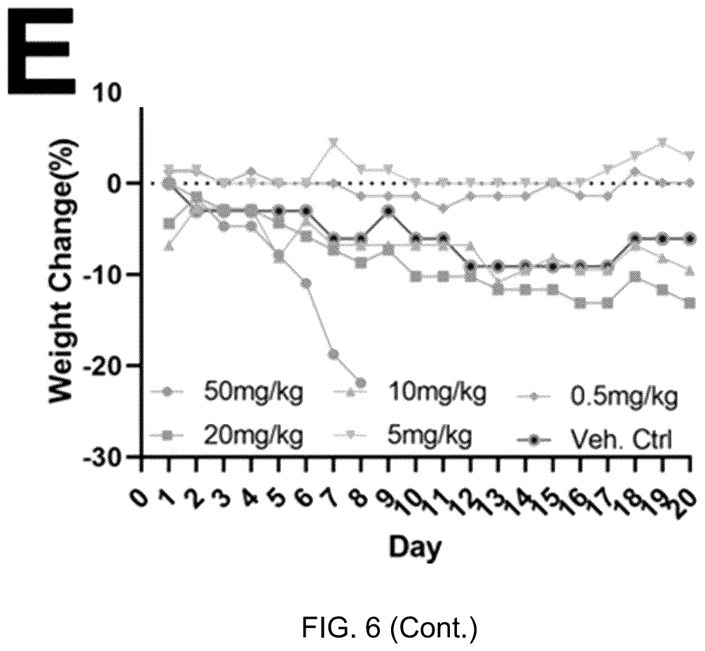

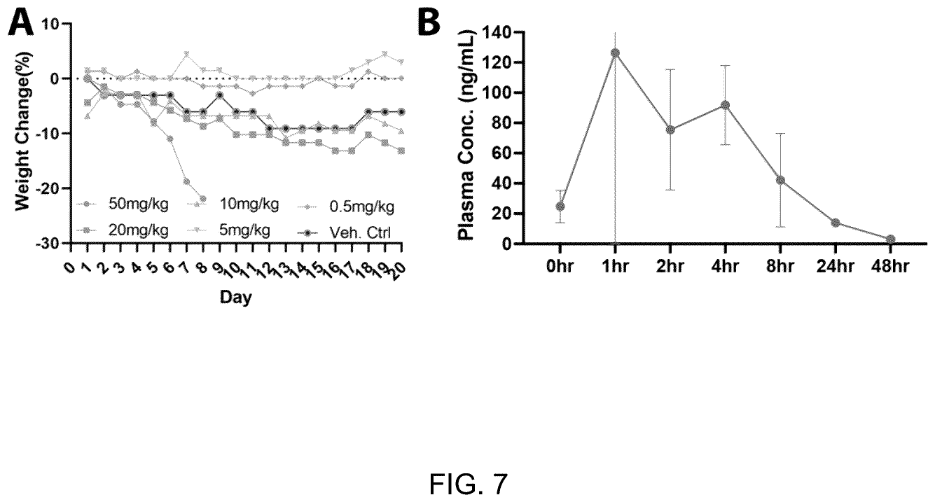

Amyotrophic lateral sclerosis, known as ALS, is a nervous system disease that affects nerve cells in the brain and spinal cord. ALS causes loss of muscle control. Mutations in TAR DNA binding protein (TDP-43) cause ALS-like signs in zebrafish. The inventors have demonstrated that foretinib induces motor neuron (MN) axonal growth and generation of new neuromuscular junctions in TDP-43 mutant Zebra fish larvae⁴⁶ (FIG. 6C) that was reflected in rescuing its swimming deficits. Thus, foretinib appears to improve neuromuscular innervation and motor control in a zebrafish model of ALS. Accordingly, a subject in need thereof may be a subject that is suffering from or has been diagnosed with ALS.

ALS results in progressive loss of motor neuron control of essential life-supporting functions like respiration and swallowing. As the inventors have demonstrated that foretinib is capable of increasing neuromuscular innervation in animals, it is believed that the administration of foretinib to, e.g., subjects with end-stage ALS, may improve respiration or swallowing and may lead to a substantial increase in the quality of life of these subjects. The ability of a physician to assess the disease stage of subjects with ALS and improvements in respiration and swallowing are considered to be routine in the art. Accordingly, methods of restoring motor control in a subject with amyotrophic lateral sclerosis (ALS) are provided and comprise administering a therapeutically effective amount of foretinib to the subject to restore motor control in the subject. As used herein, “restoring motor control” refers to the improvement of resting tremor in a subject or the ability of a subject to regain motor control of an appendage, muscle group, bodily function, e.g., respiration or swallowing after having lost such function due to the progression of ALS. The subject may have end-stage ALS. The method may improve respiration, swallowing, or both respiration and swallowing in the subject.

The inventors have demonstrated that contacting neuron with foretinib improves myelination in cultured rat sympathetic neurons (FIG. 5). MS is known to be caused primarily by destruction of myelin sheathing. It is believed that administration of foretinib to subjects that are suffering from MS will treat the MS by increasing the myelination of peripheral nerves or reducing myelin loss in peripheral nerves. Accordingly, a subject treated with the disclosed methods may have been diagnosed with multiple sclerosis (MS).

The subject may have been diagnosed with Alzheimer's disease, which is a neurodegenerative disease. Accordingly, foretinib may improve one or more sign or symptom of Alzheimer's disease in a subject through, e.g., the disclosed neuroprotective effects of foretinib. The subject may experience one or more of improved memory, cognition, and improvement in the ability to perform daily tasks.

Treating the condition or treatment includes but is not limited to ameliorating at least one symptom of the condition, reducing or slowing further progression of the condition, reducing or slowing the spread of the condition to unaffected areas. Treating a subject refers to any type of treatment that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc.

In some embodiments, “treating neuropathy” comprises reducing one or more sign or symptom of neuropathy selected from numbness, paralysis, tingling, pain, loss of sensation, sores or ulcers of the extremities, autonomic functions, e.g., sweating, control of heart rate or blood pressure, bowel or bladder voiding, and poor digestion.

### Wound Healing

The inventors have previously demonstrated that nerve-associated Schwann cells (SCs) regulate epithelial recovery in cornea, i.e., wound healing. As the innervation of the healing skin is critical for the healing process, and the inventors have demonstrated that foretinib supports peripheral innervation in a model of diabetes, it is believed that administration of foretinib will improve corneal or cutaneous wound healing in subjects with diabetes. Accordingly, methods of improving cutaneous wound healing associated with diabetic neuropathy in a subject in need thereof are provided and comprise administering a therapeutically effective amount of foretinib to the subject to improve cutaneous wound healing in the subject. Administering may comprise oral administration, intravenous administration, intradermal or subcutaneous administration, e.g., administering the foretinib to palmar or plantar tissue of the subject, or administering topically to a wound or lesion associated with the neuropathy, and concurrently administering systemically to the subject in need thereof.

### Methods of Inducing Axonal Growth

As discussed above, the inventors have discovered that the pan-kinase inhibitor foretinib surprisingly induces the growth of neurons. In particular, contacting neurons that have been deprived of the neurotrophic factor nerve growth factor (NGF) with foretinib surprisingly induces the axonal growth of the neurons. More surprisingly, contacting NGF-deprived neurons with foretinib led to greater axonal length of the neurons than neurons cultured with NGF (FIG. 6A, top row versus bottom row). Accordingly, methods of increasing the axonal length of a neuron are provided that comprise contacting the neuron with an effective amount of foretinib. The method may increase the axonal length of the neuron as compared to a neuron contacted with nerve growth factor (NGF), see, e.g., FIG. 6A.

As used herein, “contacting” refers to the cell membrane of the neuron being exposed to a compound, e.g., foretinib. Contacting may be accomplished by any method known in the art including culturing, or administering to a subject such that the compound contacts the neuron, e.g., systemically, locally, e.g., topically, intramuscularly, intrathecally, etc.

Relatedly, methods of increasing axonal length of a neuron in a subject by administering a therapeutically effective amount of foretinib to the subject to increase the axonal length of the neuron in the subject are also provided. A subject may comprise a subject that is experiencing neurodegeneration, e.g., a subject with ALS, Alzheimer's disease, MS, or diabetes. A subject may comprise a subject that has experienced a traumatic nerve injury that has severed a nerve, e.g., a spinal injury, and/or has experienced neuronal die-back degeneration.

### Foretinib

As used herein, “foretinib” refers to the compound having the formula:

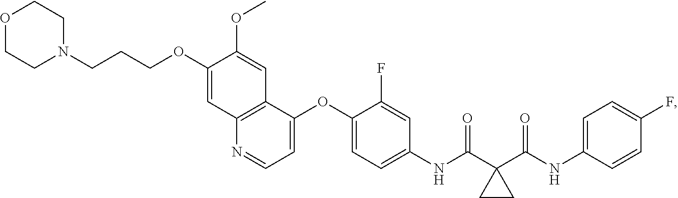 [SMILES: COc1cc2c(Oc3ccc(NC(=O)C4(C(=O)Nc5ccc(F)cc5)CC4)cc3F)ccnc2cc1OCCCN1CCOCC1]

which may also be referred to as N1-[3-Fluoro-4-({6-methoxy-7-[3-(morpholin-4-yl)propoxy]quinolin-4-yl}oxy)phenyl]-N′1-(4-fluorophenyl)cyclopropane-1,1-dicarboxamide, XL880, EXEL-2880, GSK1363089, or GSK089. Without being limited by any theory or mechanism, the inventors believe that foretinib prevents neuronal death and axonal degeneration by preventing the pro-apoptotic degradation of mitochondria and protecting mitochondrial activity. Among other activities, foretinib inhibits expression of the pro-apoptotic molecule BH3 family members, e.g., Hrk and Bax and supports the synthesis of ATP.

As used herein the term “effective amount” or “therapeutically effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with neuropathy or peripheral neuropathy.

A therapeutically effective amount, also referred to as an “effective amount,” can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances. A therapeutically effective amount may comprise about 0.5 mg/kg foretinib, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, or about 15 mg/kg foretinib. Neurons may be contacted with, e.g., about 1 nM, about 5 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 75 nM, about 80 nM, about 85 nM, about 90 nM, about 95 nM, about 100 nM, about 105 nM, about 110 nM, about 115 nM, about 120 nM, about 125 nM, about 130 nM, about 135 nM, about 140 nM, about 145 nM, about 150 nM, about 155 nM, about 160 nM, about 165 nM, about 170 nM, about 175 nM, about 180 nM, about 185 nM, about 190 nM, about 195 nM, about 200 nM, about 300 nM, about 400 nM, about 500 nM or more foretinib.

The compositions (i.e., foretinib and pharmaceutical formulations thereof) described herein may be administered by any means known to those skilled in the art, including, but not limited to, oral, topical, intranasal, intraperitoneal, parenteral, intravenous, intramuscular, subcutaneous, intrathecal, transcutaneous, nasopharyngeal, intra-lesional, intradermal, or transmucosal absorption. Thus, the compositions may be formulated as an ingestible, injectable, topical, ophthalmic topical, or suppository formulation. The compositions may also be delivered with in a liposomal or time-release vehicle. Administration of the compositions to a subject in accordance with the invention may exhibit beneficial effects in a dose-dependent manner. Thus, within broad limits, administration of larger quantities of the compositions is expected to achieve increased beneficial biological effects than administration of a smaller amount. Moreover, efficacy is also contemplated at dosages below the level at which toxicity is seen.

It will be appreciated that the specific dosage administered in any given case will be adjusted in accordance with the composition or compositions being administered, the disease to be treated or inhibited, the condition of the subject, and other relevant medical factors that may modify the activity of the compositions or the response of the subject, as is well known by those skilled in the art. For example, the specific dose for a particular subject depends on age, body weight, general state of health, diet, the timing and mode of administration, the rate of excretion, medicaments used in combination and the severity of the particular disorder to which the therapy is applied. Dosages for a given patient can be determined using conventional considerations, e.g., by customary comparison of the differential activities of the compositions described herein and of a known agent, such as by means of an appropriate conventional pharmacological protocol.

The maximal dosage for a subject is the highest dosage that does not cause undesirable or intolerable side effects. The number of variables in regard to an individual treatment regimen is large, and a considerable range of doses is expected. The route of administration will also impact the dosage requirements. It is anticipated that dosages of the compositions will improve the condition being treated by at least 10%, 20%, 30%, 40%, 50%, 60%, 70%, 80%, 90%, 100% or more as compared to no treatment.

The effective dosage amounts described herein refer to total amounts administered, that is, if more than one composition is administered, the effective dosage amounts correspond to the total amount administered. Treatment, i.e., foretinib, can be administered as a single dose or as divided doses. For example, the treatment may be administered two or more times separated by 4 hours, 6 hours, 8 hours, 12 hours, a day, two days, three days, four days, one week, two weeks, or by three or more weeks.

Foretinib or pharmaceutical compositions thereof described herein may be administered one time or more than one time to the subject to effectively improve the condition being treated, e.g., neuropathy or diabetic neuropathy. Suitable dosage ranges are of the order of several hundred micrograms effective ingredient with a range from about 0.01 to 50 mg/kg/day, preferably in the range from about 0.1 to 1 mg/kg/day. Precise amounts of effective ingredient required to be administered depend on the judgment of the practitioner and may be peculiar to each subject. It will be apparent to those of skill in the art that the therapeutically effective amount of the foretinib described herein will depend, inter alia, upon the administration schedule, whether the composition is administered in combination with other therapeutic agents, the status and health of the recipient, and the therapeutic activity of the particular composition.

In some embodiments, foretinib is administered orally. In some embodiments, foretinib is administered intravenously. In some embodiments, foretinib is administered to a targeted area of neuropathy to prevent unwanted side effects to the subject, e.g., topically.

Therefore, because peripheral diabetic neuropathy typically manifests as neuropathy in the extremities, in some embodiments, foretinib is administered to the plantar or palmar tissue intradermally, subcutaneously or topically. Foretinib may also be administered locally to any area of the body to improve neurodegeneration.

In another embodiment, the foretinib or pharmaceutical compositions comprising foretinib is administered systemically (e.g., orally or intravenously) and topically (e.g., cream, gel, lotion, etc.). In some embodiments, the foretinib or pharmaceutical compositions comprising foretinib are administered concurrently systemically and locally for improved efficacy. For example, an existing diabetic wound may be treated both locally and systemically.

### Pharmaceutical Compositions

The present disclosure also provides pharmaceutical compositions for use in treating neuropathy. The pharmaceutical compositions comprise foretinib and a pharmaceutically acceptable carrier. The pharmaceutical compositions comprise foretinib and may be formulated for topical administration, such as ophthalmic or epidermal administration. The pharmaceutical compositions, including ophthalmic formulations, with foretinib may also include an extended-release vehicle. And an extended-release vehicle may be a biocompatible polymer, dissolved in the carrier or by itself impregnated with foretinib to hold the foretinib and slowly release the drug to the subject, preferably for an extended-release period, e.g., one day, two days, three days, four days, five days, six days, seven days, or more. The biocompatible polymer may be biodegradable or non-biodegradable, depending on desired use and application schedule. Example biocompatible polymers that may be used in the disclosed formulations as an extended-release vehicle include but are not limited to poly-2-hydroxyethylmethacrylate (p-HEMA hydrogels), poly (lactic-co-glycolic) acid (PLGA), polycaprolactone (PCL), hydroxypropyl cellulose, Anecortave acetate (AnA), gelatin, and/or collagen. The inclusion of an extended-release vehicle may, in some cases, allow for less frequent application while still providing effective dosing of foretinib.

The term “pharmaceutically acceptable carrier,” as used herein, means a non-toxic, inert solid, semi-solid or liquid filler, diluent, encapsulating material or formulation auxiliary of any type. The disclosed pharmaceutical compositions may be formulated for administration by, for example, solid dosing, oral, a topical formulation, injection, inhalation (either through the mouth or the nose), implants, oral, buccal, parenteral, or rectal administration. Techniques and formulations and acceptable pharmaceutically acceptable carriers may generally be found in “Remington's Pharmaceutical Sciences”, (Meade Publishing Co., Easton, Pa.). Therapeutic compositions typically are sterile and stable under the conditions of manufacture and storage.

Suitable pharmaceutically acceptable carriers include, but are not limited to, diluents, preservatives, solubilizers, emulsifiers, liposomes, nanoparticles and adjuvants. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Additionally, pharmaceutically acceptable carriers may be aqueous or non-aqueous solutions, media, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media.

In some embodiments, the pharmaceutical composition is formulated for local delivery. Suitable additional components for topical delivery are known in the art and include creams, gels, and controlled release drug delivery materials (for example, but not limited to, e.g., PCNU, PGLA, etc.).

Compositions of the present disclosure may include liquids, lyophilized, or otherwise dried formulations and may include diluents of various buffer content (e.g., Tris-HCl, acetate, phosphate), pH and ionic strength, additives such as albumin or gelatin to prevent absorption to surfaces, detergents (e. g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the polypeptide, complexation with metal ions, or incorporation of the material into or onto particulate preparations of polymeric compounds such as polylactic acid, polyglycolic acid, hydrogels, etc., or onto liposomes, microemulsions, micelles, milamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

The compositions can be sterilized by conventional, well-known sterilization techniques. The compositions may contain pharmaceutically acceptable additional substances as required to approximate physiological conditions such as a pH adjusting and buffering agent, toxicity adjusting agents, such as, sodium acetate, sodium chloride, potassium chloride, calcium chloride, sodium lactate, and the like.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, local delivery to wounds, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

In one embodiment, both systemic (e.g., oral, intravenous, etc.) and topical administration are envisioned, for example, systemic and topic administration are performed concurrently.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3members refers to groups having 1, 2, or 3 members. Similarly, a group having 6 members refers to groups having 1, 2, 3, 4, or 6 members, and so forth.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims.

### EXAMPLES

The following examples are meant only to be illustrative and are not meant as limitations on the scope of the invention or of the appended claims.

### Methods

### Study Design

The primary objective of these experiments was to determine the effect of oral Foretinib on cutaneous innervation density in an experimental diabetic neuropathy. The numbers of animals needed for these experiments were determined by power analysis based on preliminary experiments assessing the cutaneous innervation density in diabetic and non-diabetic mice. A 50% increased nerve fiber density compared to the diabetic control was considered scientifically relevant, requiring a minimum of n=5 animals per group to achieve a power of 0.9 when assuming a standard deviation (SD) of 0.01 for all groups (normally distributed, two-sided). Animals were randomly allocated to experimental groups prior to induction of diabetes and the investigators were blinded during outcome assessments. Two experimental animals allocated to the Foretinib treated group were excluded from the experiment after not responding to the STZ injections. No outliers were excluded from the analysis. After five weeks, the experiment was terminated because 4 of 8 animals reached a predefined humane endpoint of greater than 15% weight loss compared to their pre-diabetic weight. The scheduled experimental endpoint was 12 weeks post-STZ injection.

### Experimental Animals

A total of 25 adult (24-28g), transgenic mice with a genetic black six background were included. The B6.Cg-Tg(Thy1-YFP)16Jrs/J mice expressed yellow fluorescent protein (yfp) under the thy-1 promotor and therefore had yellow fluorescent neuronal cells which enabled dye-free fluorescent nerve fiber imaging. All animals were housed in a central animal care facility with fresh water and pellet food ad libitum. A constant room temperature (22° C.) and a circadian rhythm of 12 h per 24 h illumination were automatically maintained. All procedures were performed in strict accordance with the National Institutes of Health guidelines, the Canadian Council on Animal Care (CCAC) and were approved by the Hospital for Sick Children's Laboratory Animal Services Committee.

### Induction and Monitoring of Diabetes

In total, 20 mice were injected with 100 mg/kg bodyweight of streptozotocin (STZ, S0130, Sigma Aldrich) in 0.1M citrate buffer (pH 4.5) on two consecutive days respectively, to induce diabetes type 1²⁷. Prior to the first injection and in weekly intervals thereafter, all animals were weighted and underwent tail prick blood sugar measurements using a clinical grade monitoring system (Contour One, Ascensia Diabetes Care, Mississauga, Ontario) as previously described. Mice were considered diabetic when blood glucose was at least 15 mmol/L (270 mg/dl).

### Foretinib and Vehicle Treatments

Prior to diabetes induction the mice were block randomized into experimental groups (n=10 Foretinib treatment, n=5 vehicle treatment, n=5 non-treated diabetic control). An additional 5 non-diabetic animals served as a healthy control. Foretinib treatments started 1-week post-diabetes induction. Foretinib was prepared fresh daily by dissolving 6 mg Foretinib in 1 ml of 1% (Hydroxypropyl)-methyl cellulose (09963, Sigma Aldrich) and 0.2% sodium dodecyl sulfate (L3771, Sigma Aldrich) in sterile H₂O. A dose of 30 mg/kg body weight Foretinib was administered via daily gavage using a reusable steel feeding needle, (18 G, curved, 50 mm, Fine Science Tools GmbH, Heidelberg, Germany). Vehicle treated mice received 1% (Hydroxypropyl)-methyl cellulose and 0.2% sodium dodecyl sulfate in sterile H₂O daily in similar fashion.

### Assessment of Intraepidermal Nerve Fiber Density

Five weeks post diabetes induction, animals were sacrificed and 3×1 mm full thickness plantar skin flaps from both hind paws were harvested. One skin sample of each animal was used for longitudinal cryosections, and the contralateral sample underwent optical tissue clearing. Skin flaps were straightened and fixed by immersion in precooled 4% paraformaldehyde (PFA) at 4° C. for 24 h in the dark. For cryosectioning the samples were cryoprotected in 4% PFA with 30% sucrose for 2-5 days at 4° C., then embedded in Tissue Freezing Medium (Electron Microscopy Sciences, Hatfield, USA) and frozen at −80° C. in for a minimum of 24 hours. Then the tissue blocks were mounted in a cryostat microtome (CM3050S, Leica Microsystems, Wetzlar, Germany) and cut in 50 μm longitudinal sections. The tissue sections were mounted on Superfrost Plus microscope glass slides (Fisher Scientific, Pittsburgh, PA, USA), immunostained against the neuronal marker beta 3-tubulin with a nuclear counterstaining (DAPI). The following antibodies were used: rabbit anti-beta 3 tubulin (18207, Abcam, Cambridge, UK; 1:500 dilution) ith goat anti-rabbit Alexa Fluor 488 conjugated secondary antibody (ab150077, Abcam, Cambridge, UK; 1:1000) and DAPI (D1306, Thermo-Fisher Scientific, Massachusetts, USA, 1:1000). Then samples were imaged with a 1 μm z-step interval using a Leica SP8 Lightning confocal microscope (DMI8, Leica Microsystems, Wetzlar, Germany), equipped with Leica LAS software, a Hybrid detector (HyD), a p.co Edge 5.5 camera (PCO AG) and a 20×/0.75 (W) objective. For data processing, we used Arivis Vision 4D (version 3.0, Arivis AG, Rostock, Germany) or Leica LAS (Leica Microsystems, Wetzlar, Germany) for three-dimensional tile stitching and Imaris (Version 9.5.1, Bitplane AG, Zurich, Switzerland) for image segmentation and quantitative morphometric analyses. Intraepidermal nerve fiber density (IENDF) was determined as fibers per mm by a blinded investigator by scrolling through the stitched z-stacks over a 2.5 mm long skin segment.

### Three-Dimensional Assessment of Cutaneous Innervation

For three-dimensional analysis, full thickness skin samples were straightened and fixed by immersion in precooled 4% paraformaldehyde (PFA) at 4° C. for 24 h in the dark, washed in 1× PBS and subsequently cleared using a modified FDISCO protocol 28. Briefly, the tissue was immersed in an ascending series of precooled (4° C.) pH 9.0 adjusted Tetrahydrofuran (186562, Sigma-Aldrich) double distilled water solutions (50 vol %, 75 vol %, 3×100 vol %) and a subsequent step in 100% Dichloromethane (270997, Sigma-Aldrich) at room temperature for dehydration and delipidation²⁸. Then the specimen was immersed in Dibenzyl ether (108014, Sigma-Aldrich) overnight at 4° C. in the dark, with two changes of the solution, to match the refractive index of the tissue with the microscope objective. Specimens were imaged in Cell

Imaging Dishes with a 1.0 μm cover glass bottom (Eppendorf AG, Hamburg, Germany) using the Leica SP8 Lightning confocal microscope (DMI8, Leica Microsystems, Wetzlar, Germany) as described above. Cutaneous nerve fibers were semiautomatically traced using batch processing with consistent fluorescence intensity thresholds across the entire data set. Volumetric nerve fiber density was determined as total nerve fiber volume per skin tissue volume for an 830×830×200 μm region of interest.

### Lower Extremity Nerve Histomorphology

Five weeks post-diabetes induction a 5 mm sciatic nerve segment was harvested 5 mm proximal to the sciatic bifurcation and a 5 mm sural nerve segment was harvested at the ankle level. Samples were gently straightened, immersed in 2.5% glutaraldehyde fixative (G6257, Sigma Aldrich)/0.1 M sodium cacodylate trihydrate (C0250, Sigma Aldrich) overnight at 4° C. and postfixed in 2% osmium tetroxide (75632, Sigma Aldrich) for 2 h and dehydrated in ascending ethanol series. Then the nerves were embedded in epoxy (45345, Sigma Aldrich), sectioned into 1-μm cross-sections (ultramicrotome EM UC7, Leica Microsystems) and imaged (Axiovert 200M, Carl Zeiss Microscopy GmbH, Jena, Germany) using a 63×/1.4 oil objective. A custom-trained deep learning model based on the open-source software AxonDeepSeg²⁹ determined axon diameter and myelin sheath thickness and g-ratio for entire nerve cross sections.

### Statistical Analysis

We used GraphPad Prism 9 (GraphPad Software, San Diego, California, USA) for statistical analysis. Descriptive statistics were calculated, and means are expressed with standard deviations (±SD) if not indicated otherwise. To test for normality of continuous variables, we used normal quantile plots and Shapiro-Wilk tests. For between group comparisons, one-way analysis of variance (ANOVA) or a mixed effect model with Geisser-Greenhouse correction were performed with Tukey's multiple comparison tests. A significance level of 5% was used (p<0.05). Selected images in this publication are created with BioRender.com.

### Example 1: Foretinib Reduces Cutaneous Nerve Fiber Loss Due to Diabetic Neuropathy

In the following example, the inventors describe the use of the pan-kinase inhibitor foretinib in the treatment of neuropathy in an experimental animal model of diabetic neuropathy. In addition, the inventors refer to the published manuscript Daeschler et al., “Foretinib mitigates cutaneous nerve fiber loss in experimental diabetic neuropathy” Sci Rep. 2022 May 19;12(1):8444, which is incorporated herein by reference in its entirety.

### Results

### Phenotype of the Diabetic Mouse Model

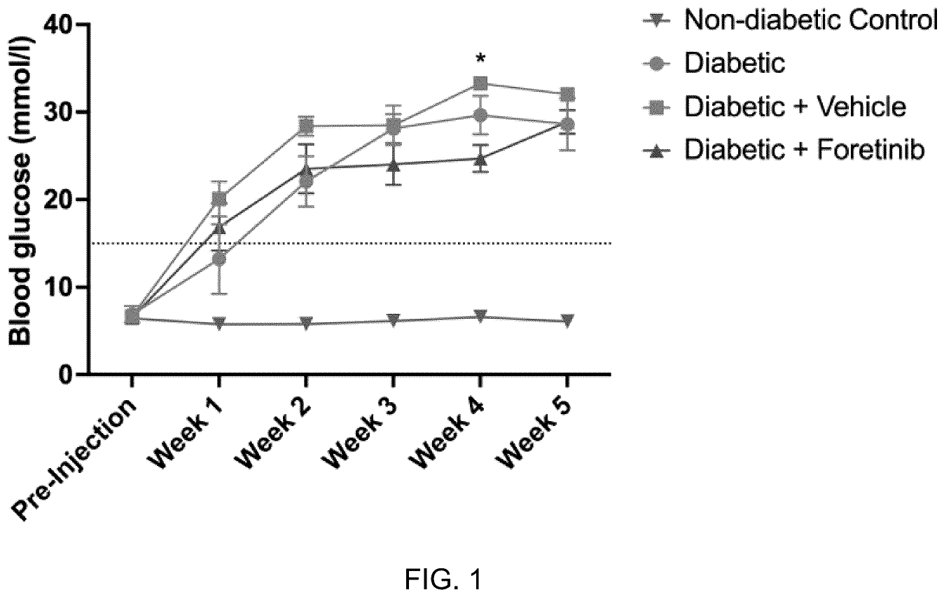

In total, 18 of 20 streptozotocin-injected mice developed a stable diabetic state within 1 to 2 weeks post-injection, which was defined as blood glucose levels greater than 15 mmol/l (270 mg/dl). Two non-responding mice were excluded from the experiment. No antidiabetic drugs were used. The blood glucose concentrations in the non-diabetic control mice remained within physiological levels throughout the entire experimental period (FIG. 1).

### Foretinib Mitigates Cutaneous Nerve Fiber Loss in Experimental Diabetic Neuropathy

Sensorimotor diabetic neuropathy affects nerve fibers in a length-dependent fashion, with loss of terminal cutaneous nerve fibers of the distal-most parts of the lower extremities occurring first. Five weeks post-STZ-injection we therefore harvested a plantar full thickness skin grafts from both hind paws to assess cutaneous innervation. We first used conventional cryosectioning to obtain longitudinal cross sections of the plantar skin and determine the intraepidermal nerve fiber density (IENFD). Compared to non-diabetic mice, the IENFD was significantly decreased by approximately 25% in all diabetic groups, with no significant differences among them (FIGS. 2A to 2C and 2H).

However, observations in cryosections are largely limited to two dimensions and thus do not necessarily reflect the three-dimensional morphology of the cutaneous nerve fiber plexus. We therefore used optical tissue clearing to render the skin grafts of the contralateral plantar surface transparent, allowing confocal assessment of the cutaneous innervation in three dimensions. Using fluorescence intensity-based image segmentations we traced nerve fiber bundles from the deep dermis up to their terminal epidermal branches to determine the volumetric nerve fiber density within the skin graft (FIGS. 2D to 2G). In control mice, epidermal nerve fibers arose from a dense three-dimensional network of interconnected subepidermal nerve fiber bundles originating from larger nerve branches in the deep dermis and subcutis (FIG. 2D). In non-treated and vehicle-treated diabetic mice, we observed a significant subepidermal nerve fiber loss primarily affecting thin, horizontally oriented nerve fiber bundles in the superficial dermis (FIGS. 2E and 2G). This reflected in significantly reduced cutaneous nerve fiber densities of 44.9% (non-treated) and 37.8% (vehicle-treated) of the non-diabetic reference respectively. In contrast, the subepidermal nerve fiber plexus in Foretinib-treated mice was largely preserved resulting in a significantly greater cutaneous nerve fiber density equaling 67.3% of non-diabetic reference compared to non-treated and vehicle treated diabetic mice, though thinning and loss of dermal nerve fiber bundles were still evident (FIG. 2F). This suggests that Foretinib mitigated the cutaneous nerve fiber loss in mice suffering from streptozotocin-induced diabetes (FIG. 2I).

### Foretinib Does Not Affect Proximal Nerve Fiber Morphology

Advanced diabetic neuropathy may result in structural alterations of lower extremity nerves including myelinated and unmyelinated nerve fiber loss and focal de-and remyelination²¹. To determine the effect of Foretinib on nerve fiber histomorphology, we assessed cross sections of the sciatic nerve approximately 5 mm proximal to its bifurcation and the sural nerve at the level of the ankle (FIGS. 3A-F and M-P). In agreement with the literature, we observed no alterations of axon diameter (FIGS. 3G and J), myelin sheath thickness (FIGS. 3H and K) and g-ratio (FIGS. 3I and L) between healthy mice and mice five weeks post-diabetes induction. Neither the Foretinib nor the vehicle treated groups differed from the non-treated diabetic group. This indicates that the Foretinib treatment did not affect peripheral nerve histomorphology and confirmed that diabetes-associated alterations of nerve fiber morphology within peripheral nerves are usually not observed within five weeks post diabetes induction in this STZ mouse model.

### Potential Side Effects of Foretinib

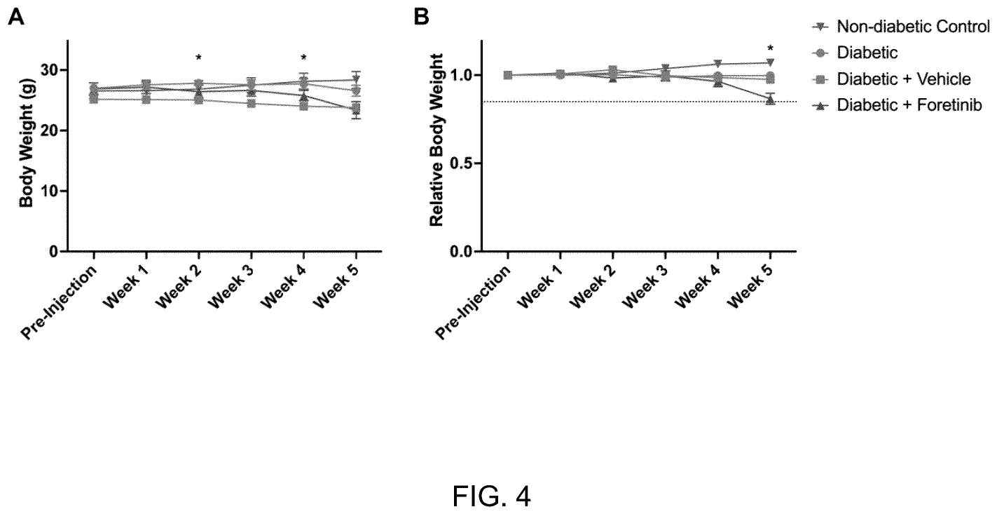

Foretinib treated animals received 30 mg/kg body weight Foretinib orally via daily gavage²². After four weeks of daily treatment, we observed signs of increased distress in mice that had received Foretinib including lethargic behavior, ruffled fur, and weight loss (FIG. 4A).

In week five, 50% of animals in this group reached a humane endpoint of greater than 15% weight loss compared to their pre-diabetic weight (FIG. 4B). This was despite supportive care such as softened chow²³ and did not occur in the other experimental groups, suggesting adverse side effects of Foretinib. The experiment was therefore terminated after 5 weeks instead of 12 weeks.

### Discussion

Diabetic neuropathy is the most common cause of neuropathy, resulting in progressive degeneration of terminal nerve fibers associated with loss of sensation, paresthesia, and persistent pain^{3,4}. Presently available treatment strategies focus on pain control but are unable to prevent or mitigate the axonal loss in diabetic patients¹⁶. Here we demonstrate that the pan kinase inhibitor Foretinib rescues subepidermal nerve fibers in experimental diabetic neuropathy, without affecting proximal nerve fiber morphology. However, the daily oral treatment of 30 mg/kg Foretinib for four weeks resulted in significantly greater weight loss and poor body condition compared to mice receiving a vehicle.

In diabetic individuals, neurons are exposed to chronic hyperglycemia. The consequent cellular nutrient overload leads to mitochondrial dysfunction which is hypothesized to cause ATP depletion in terminal axons and thereby contribute to their pruning^{14,15}. We have previously shown that Foretinib prevents axonal die-back mechanisms in several pathological conditions, including trophic deprivation in sensory and sympathetic neurons in vitro by preserving mitochondrial integrity and thereby axonal energy supply, and preventing activation of the apoptotic signaling cascades¹⁷. To determine the therapeutic potential of this drug for diabetic neuropathy we used the well-described STZ mouse model²⁴⁻²⁶. Five weeks post diabetes induction we compared cutaneous innervation and peripheral nerve histomorphology of diabetic mice that received 30 mg/kg/day Foretinib orally with non-treated and vehicle treated diabetic controls. This treatment regimen was based on previous work reporting a potent treatment response in rodent anti-tumor studies and therefore indicating systemic availability of bioactive levels of Foretinib²².

Five weeks after diabetes induction, all diabetic groups showed a comparable 25% reduction in epidermal nerve fiber density in the plantar skin compared to healthy mice. This is in agreement with previously reported observations in this experimental model of diabetic neuropathy²⁴. However, in two dimensional histological cross sections morphological changes of the subepidermal plexus cannot be adequately evaluated. We therefore used optical tissue clearing to assess the skin innervation in three-dimensions and observed a significantly higher dermal nerve fiber density with more intact nerve fiber bundles in Foretinib treated mice as compared to non-treated and vehicle treated controls. As intraepidermal nerve fibers represent the distal most aspects of lower extremity sensory axons, the dermal nerve fiber plexus includes the preceding axonal segments. Hypothesizing a partial rescue effect of Foretinib on dysfunctional axonal mitochondria in diabetic neurons, the energy deficit in Foretinib-exposed axon terminals may be less pronounced which may have contributed to the partial preservation of axonal length in these mice. Though, the axonal die-back degeneration was not completely prevented by the applied Foretinib treatment regimen. We also determined the nerve fiber morphology in more proximal segments, within the sural nerve at the ankle level and the sciatic nerve proximal to its bifurcation. As expected, we did not observe any changes in proximal nerve fiber morphology after five weeks of diabetes, as such changes usually occur at later time points in more advanced diseases stages²⁴. Future studies may investigate longer treatment and disease durations to determine the long-term effects of Foretinib in diabetes.

This study aimed at a proof of concept and therefore utilized comparably high doses of Foretinib to reliably achieve therapeutic drug levels in the target tissue. This dosing and the daily gavaging turned out to be demanding for the mice and resulted in a poor body condition after five weeks of treatment in some animals. These side effects associated with oral daily Foretinib treatment presently represent a major limitation for the translatability of our results into clinical studies. Therefore, future studies need to determine whether lower systemic doses or local drug delivery approaches may achieve similar therapeutic effects with less systemic exposure. Previous anti-cancer studies with Foretinib may help to determine minimal systemic doses necessary to achieve bioactive drug levels after the first pass effect. Further, implantable drug delivery devices may allow for extended treatment duration an overcome the need for a daily gavage in experimental rodent models.

In conclusion, our results demonstrate a partial recue effect of the pan-kinase Inhibitor Foretinib on cutaneous nerve fibers in experimental diabetic neuropathy. Future studies may help to define a sustainable treatment regimen and thereby allow patients to take advantage of this neuroprotective drug in chronic neurodegenerative diseases like diabetic neuropathy.

### Example 2—Treatment of Amyotrophic Lateral Sclerosis With Foretinib

In one example, a subject suffering from amyotrophic lateral sclerosis (ALS) may be treated with foretinib. A subject may be administered a therapeutically effective amount of foretinib. The foretinib may suitably be administered in vivo by any route that is indicated by the particular treatment needs of the subject, e.g., orally or intravenously. Signs and symptoms of ALS may be reduced by the in vivo administration of foretinib. Treatment may be administered daily, every other day, every third day, or on a schedule as determined by the patient's progress, pursuant to a physician's decision. It is anticipated that the subject may experience an increase in quality of life associated with reduction in signs or symptoms of ALS as compared to an untreated subject. Methods of measuring reductions in signs and symptoms of ALS are known in the art, e.g., measuring muscle twitches in the arm, leg, shoulder, or tongue, muscle cramps, tight and stiff muscles (spasticity), muscle weakness affecting an arm, a leg, the neck, or diaphragm, slurred and nasal speech, difficulty chewing or swallowing. In addition, as the inventors have demonstrated that foretinib improves motor nerve innervation in a model of ALS (FIG. 6D), it is believed that the administration of foretinib to subjects with ALS, e.g., end-stage ALS, may improve breathing or swallowing in the subjects, which may be measured by routine methods known in the art.

### Effectiveness of Foretinib in Models of Amyotrophic Lateral Sclerosis (ALS)

The first mutation identified that causes ALS was in SOD1, and mice engineered to overexpress the G93A-SOD1 mutation develop MN disease, similar to human ALS. More than 20 genes have since been identified with ALS-associated mutations, many of which are de novo mutations present in sporadic ALS patients². TAR DNA binding protein of 43 kDa (TDP-43) was identified as a gene mutated in ALS and, importantly, almost 90% of ALS cases (both wt and mutant TDP-43) are characterized by the presence of aggregates that contain insoluble, misfolded cytoplasmic TDP-43. The analysis of these and other ALS-associated genetic pathways has revealed several molecular mechanisms that may contribute to the pathogenesis of ALS, such as axonal transport defects, toxic protein aggregation, impaired nucleocytoplasmic transport, inflammation, and mitochondrial deficiency. More recently several genes linked to ALS are involved in RNA transport, splicing and translation, suggesting that deficiencies in RNA metabolism contribute to MN degeneration.

### Mitochondria

Mitochondrial dysfunction is one of the earliest pathophysiological events in ALS. Disruption of mitochondrial structure, dynamics, bioenergetics, and calcium buffering has been extensively reported in ALS patients and model systems and is involved in disease pathogenesis. Structurally altered and aggregated mitochondria, with a swollen and vacuolated appearance, were some of the first changes observed in the MN of postmortem tissues from ALS patients. Several proteins that have been linked to familial and sporadic ALS, including SOD1 and TDP-43, have been shown to interact with mitochondria. Interestingly, mutations in these two proteins directly interfere with mitochondrial respiration and ATP production and cause oxidative stress. These observations suggest mitochondria are a potential therapeutic target of ALS.

### Foretinib

Foretinib is phase II study, clinically safe anticancer multi-kinase inhibitor drug. Using a drugs screen assay, we discovered that Foretinib, with a high efficiency, prevents neuronal death and axonal degeneration in several pathological in vivo and in vitro conditions, including cultured MNs from Sod1^{G93A} mice. Foretinib mediates its neuroprotective effect via protecting mitochondrial integrity and activity. In our recent studies we demonstrated that Foretinib has a significant inhibitory effect on the progression of diabetic peripheral neuropathy in mice that, like ALS, is associated with mitochondrial stress. Preliminary observations showed that Foretinib, along with preventing axonal die-back, induced axonal growth of nerve growth factor (NGF)-deprived rodent sympathetic neurons in vitro (FIG. 6A). Foretinib treatment also prevented the degeneration of MNs derived from human induced pluripotent stem cells (iPSCs) in vitro, and in TDP-43 mutant Zebra fish larvae facilitated axonal growth and neuromuscular junction (NMJ) formation that reflected in re-gaining of sweeping capability of the, otherwise paralyzed, mutant larvae (FIG. 6B-D and not shown). It is expected that in ALS, Foretinib will preserve mitochondrial integrity and activity, thereby inhibiting death of motor neurons and axonal degeneration.

The three FDA approved medications for ALS, Riluzole, Edaravone and Relyvrio, only extend life by 2-3 months. Foretinib is a clinically safe compound with potent neuroprotective activity, including of motor neurons, as observed in several in vitro and in vivo studies. This project holds promise for providing the preclinical basis for a novel, safe treatment for ALS.

Foretinib has an established safety and pharmacokinetic profile and has been successfully evaluated in previous phase II cancer trials. The unexpected discovery of Foretinib's potent neuroprotective and neurotrophic activity opens a new opportunity for patients with ALS. Foretinib's established FDA safety record would enable rapid progression to clinical trials.

We have further identified the effect of Foretinib treatment on survival and physiology of MN in ALS. Foretinib induced survival of trophic factor-deprived wt and Sod1^{G93A} cultured mouse MN. We have also demonstrated that Foretinib rescued Thapsigargin-and Tunicamycin-induced death of human iPSC-derived cultured MNs (FIG. 6A,B). Preliminary experiments suggested that Foretinib induces motorneuron (MN) axonal growth and generation of new neuromuscular junctions in TDP-43 mutant Zebra fish larvae (FIG. 6C) that was reflected in rescuing its swimming deficits (not shown). These data demonstrate Foretinib's in vivo neuroprotective activity in an ALS model. Taken together, these results highlight Foretinib's therapeutic potential in human ALS. We defined intraperitoneal (IP) daily 5 mg/kg body weight as the maximal tolerable dose of Foretinib that did not affect mice behavior and body weight (FIG. 6E). We are currently working on defining pharmacokinetics (PK) and the body's organ distribution of Foretinib in wt mice.

In Sod1^{G93A} mice, detectable degeneration of MN can be observed as early as 3 months of age, which is reflected in detectible behavioral abnormalities that get progressively worse. In this set of experiments, we will define the effect of Foretinib treatment on (i) preventing degeneration of MN in the early stages and (ii) treating progressing degeneration of MN in the later stage of ALS. Therefore, mice will be injected IP daily with Foretinib from the age of (i) 1 month and the age of (ii) 3 months, respectively, until 5 months (which is about Sod1^{G93A} mice life span). Sod1^{G93A} Foretinib-treated mice will be compared with Sod1^{G93A} vehicle-only and wt Foretinib-treated mice. The mice will be assessed using: 1) morphology of neuromuscular junctions and MN counts and 2) motor performance and life span of mice in every experimental group that will not be sacrificed by 5 months of age. Monthly standardized behavioral observations will be performed, starting from the age of 2 months until end stage ALS, defined as CS4 (Clinical Score 4; i.e., complete loss of hindlimb function). As a complimentary approach, the effect of Foretinib treatment on the survival of human iPSC-derived MN, comparing between wt and SOD1^{L144F}, will be assessed in culture. We anticipate that the treatment with Foretinib will inhibit MN degeneration in vitro and in vivo, leading to improved motor behavioral, life span and neuronal survival.

We expect to demonstrate that Foretinib halts the progression of ALS. Foretinib's established safety record would enable rapid translation into clinical trials. Further, the data from these studies may establish reliable biomarkers for ALS, which could have important use for clinical trials.

### Example 3—Treatment of Multiple Sclerosis With Foretinib

In one example, a subject suffering from multiple sclerosis (MS) is administered a therapeutically effective amount of foretinib. The foretinib may suitably be administered in vivo by any route that is indicated by the particular treatment needs of the subject, e.g., orally or intravenously. Signs and symptoms of MS may be reduced by the in vivo administration of foretinib. Treatment may be administered daily, every other day, every third day, or on a schedule as determined by the patient's progress, pursuant to a physician's decision. It is anticipated that the subject may experience an increase in quality of life associated with reduction in signs or symptoms of MS as compared to an untreated subject, including prevention of further loss of myelination of the peripheral nerves, or an increase in myelination of peripheral nerves. Methods of measuring reductions in signs and symptoms of MS are known in the art, e.g., measuring fatigue, gait difficulties, spasticity, vision problems, dysesthesia, numbness or tingling, weakness, vertigo or dizziness, cognitive changes, pain or itching.

### Example 4—Improving Cutaneous Wound Healing With Foretinib

A subject suffering from cutaneous wounds associated with diabetes may be administered a therapeutically effective amount of foretinib to treat the wound. The foretinib may suitably be administered by any route that is indicated by the particular treatment needs of the subject, e.g., orally, intravenously, topically, or any combination of orally, intravenously, and topically. Wound healing may be improved by the administration of foretinib. Treatment may be administered daily, every other day, every third day, or on a schedule as determined by the patient's progress, pursuant to a physician's decision. It is anticipated that the subject may experience improved time to wound closure, improved pain, reduced risk of infection, and/or reduced likelihood of requiring amputation as compared to an untreated subject as the inventors have demonstrate that foretinib antagonizes neurodegeneration caused by diabetes and improved cutaneous innervation is correlated with improved wound healing. Methods of measuring the rate of wound healing and the quality of the healing process are known in the art and are routinely evaluated by a physician and may include standard sensory testing.

Diabetic peripheral neuropathy and neuropathic wounds may be treated with foretinib. To date, 1 in 11 adults, or 463 million people worldwide, have diabetes, a number that has more than tripled over the past two decades, with a substantial prevalence of type 2over type one diabetes. Of those individuals, up to 50% eventually develop a neurodegenerative disease termed peripheral diabetic neuropathy, with loss of sensation, paresthesia, and, in one-third of patients, persistent pain. Individuals with diabetic peripheral neuropathy (DPN) are also very likely to be affected by major chronic wound healing deficits in the skin and cornea, with estimates ranging from 8-25%. Notably, the presence of DPN is associated with a seven-fold higher risk of developing a foot ulcer in comparison with non-neuropathic diabetic individuals, linking these severe complications. Chronic wounds, which can lead to limb amputation, are a significant cause for morbidity and mortality for this patient population. DPN in cornea often leads to the corneal opacification and, eventually, vision loss. Members of the military who develop diabetes during active duty are referred for possible medical discharge or retirement. Medical surveillance of the U.S. military indicates that the incidence of all types of diabetes is similar to that of the civilian population (1.9 vs. 1.6 cases per 1,000 person-years) despite weight and fitness standards. Per the US Department of Veteran Affairs, 25% of VA patients have diabetes. Foot ulcers precede 85% of diabetes-related amputations, according to the American Podiatric Medical Association. In 2019, more than 9,000 veterans underwent lower extremity amputations, with more than 3,000 of those involving the loss of a foot due to wound healing complications. Despite the massive consequences of DPN and wound healing deficits, current treatment strategies fail to prevent or reverse axonal loss or accelerate wound closure in diabetic patients.

Neuronal hyperglycemia impairs mitochondria, leading to exhaustion of the ATP supply in distal axons. Cutaneous Schwann cells (SC) are also very sensitive to hyperglycemia; diabetes stimulates deviations in SC glucose and lipid metabolism, cell signaling patterns, gene expression, and cell structure. These pathophysiological changes to SC, as in neurons, are associated with mitochondrial malfunctioning that occur prior to degeneration of axons.

Impaired SC mitochondrial metabolism alters long term axon function and survival. However, the safe-in-human pan-kinase inhibitor foretinib is a therapeutic candidate that prevents axonal die-back in peripheral neurons by rescuing mitochondrial survival and activity, thereby preventing energy depletion and cytoskeletal degradation. More recent findings demonstrate that foretinib inhibits early axonal degeneration in streptozotocin-induced type 1 diabetic mice. Further, we disclose herein that foretinib improves axonal myelination (FIG. 5) and axonal growth (FIG. 6A) in culture. We expect foretinib to rescue cutaneous axons and SC in early diabetes and will induce axonal regeneration in the chronic stage of type 2 diabetes, enhancing sensation and wound healing.

Determine the effect of foretinib on axonal and glial survival in mouse genetic model of type 2 diabetes. Foretinib is expected to protect terminal axons and the ensheathing SC and/or induce their regeneration during early and chronic hyperglycemic conditions, respectively, in vivo. To test this hypothesis, we will administer foretinib in a mouse genetic model of type 2 diabetes (db/db) that develops diabetic neuropathy and dermal wound healing deficits.

Determine the effect of foretinib on dermal and corneal wound healing during diabetes. Foretinib may improve diabetes-associated wound healing deficits in vivo. To test this hypothesis, we will compare the rate and extent of wound healing in foretinib-treated and untreated control db/db mice.

Determine the effect of foretinib on axonal and glial survival in mouse genetic model of type 2 diabetes. Besides axonal die-back, DPN is associated with axonal demyelination and disruption of nodes of Ranvier. These pathological processes are associated with mitochondrial activity deficits in SC. Foretinib is a strong neuroprotective agent that preserves mitochondrial integrity and rescues early DPN in streptozotocin-induced type1 diabetes mouse models. Here we will test the effect of foretinib on db/db mice, which develop type 2 diabetes-induced DPN. Studies of primary wt neuronal/SC co-cultures demonstrated that foretinib significantly improved axonal myelination (FIG. 5).

It is expected that Foretinib will protect against the diabetes-induced loss of terminal axons and ensheathing SC. In our previous study, 30 mg/kg of foretinib was administered via daily gavage for a course of 5 weeks. Although reported as non-toxic, at this dose the drug, along with a strong neuroprotective effect, caused lethargic behavior, ruffled fur and weight loss requiring early termination of the experiment. Due to the substantial prevalence of type 2 diabetes, we will examine the effect of foretinib treatment on type 2 diabetic mice (db/db). To define the maximal tolerable dose, we treated wt mice by daily intraperitoneal (IP) injection of several various dosages of foretinib for 20 days, while assessing the mice weight and behavior. Daily 5 mg/kg IP was the maximal dose that did not affect weight or behavior (FIG. 7A). Next, to define the pharmacokinetic (PK) characteristics of the drug, we performed a mass spectrometry analysis of blood samples from wt mice treated with 5 mg/kg (n=3 mice per dose per time point). We observed that IP injection of 5 mg/kg of foretinib generated nearly 100 ng/ml blood concentration during first 4 hours, declining by 4 ng/ml/hour (FIG. 7B). The concentration of 100 ng/ml (160 nM) is comparable to the effective neuroprotective concentration used in primary culture of sympathetic and sensory neurons (>100 nM).

db/db mice typically develop diabetes at 4 weeks of age and exhibit a severe neurological phenotype by 12-16 weeks of age. To examine the effect of foretinib on the axonal and glial survival during diabetes, following the PK test results, we will treat db/db mice with 5 mg/kg/d for 12 weeks, starting at 4 weeks of age. Vehicle-only db/db and foretinib-treated non-diabetic db/+mice (n=8 per group) will be used as negative and positive controls, respectively. At the age of 16 weeks (after 12 weeks of treatment), full thickness plantar skin from both hind paws and sciatic nerve segments will be harvested and analyzed. The analysis will include immunostaining for bIII-tubulin, S100b, GFAP, p75^{NTR} to quantify neuronal and glial cell populations. Intraepidermal nerve fiber density will be compared between the three experimental conditions, as described. Sciatic nerves will be immunostained for myelinating and non-myelinating SC, nodes of Ranvier and paranodal junction markers (MBP, MAG, Na⁺ channels, Gldn, Caspr), and the presence and morphology of myelinating and non-myelinating SC will be assessed by electron microscopy. The latter will be supported by electrophysiological analysis of peripheral nerves, using our unique synaptic transistor-based sensor framework (an ad hoc synaptic junction device) to track changes in neuronal conductivity in vivo throughout the entire experiment. In previous studies we were able to detect reduction of conduction velocity and postsynaptic signal amplitude in db/db mice during disease progression from 7 to 30 weeks of age. We will use this electrodiagnostic approach to measure the extent to which foretinib rescues neuronal function. We anticipate that foretinib will rescue DPN. We also anticipate this rescue will occur without observing toxicity in foretinib-treated mice.

Complementary Analysis. Loss of corneal nerve fibers is among the first morphologic manifestations of DPN, sometimes even preceding loss of nerve fibers in the skin. Loss of corneal sensory innervation (Neurotrophic Keratopathy) causes blindness due to repetitive corneal epithelial injuries and failure to heal. To define the effect of foretinib on the condition of corneal nerves, we will harvest and analyze corneas from mice, as above. We will immunostain the tissues for neuronal, SC and node of Ranvier markers to define the effect of the treatment on corneal neurons, SC, and their myelinating potential. We anticipate that the treatment with foretinib of the diabetic mice will rescue corneal innervation, SC numbers and myelination.

Previously, we used neuronal compartmented chambers (that allow a separate treatment of neuronal cell bodies and their axons (FIG. 6A)) to define the neuroprotective effect of foretinib on axonal survival by substituting nerve growth factor (NGF) with the drug in the axonal compartment containing cultured primary sympathetic neurons. In our current preliminary study, we repeated this experiment on the cultured sympathetic neurons with NGF withdrawn, while adding foretinib to the central compartment (containing cell bodies) and one of the side (axonal compartments) (FIG. 6A). Surprisingly, this treatment not only supported cell bodies and axonal survival 48 hours post NGF withdrawal, but induced axonal growth, similarly to the NGF containing cultures (FIG. 6A). This preliminary observation (n=2), in addition to the neuroprotective effect of foretinib, suggests a neurotrophic effect of the compound, via an unknown mechanism. Here we will test the hypothesis that foretinib will induce regeneration of degenerated distal axons in chronic diabetic mice.

To define the potential effect of foretinib on regeneration of cutaneous neurons, we will IP treat diabetic mice in an advanced stage of DPN, after the degeneration of the distal axons has already occurred. We will assess the effect of the treatment on myelination and cutaneous axonal regeneration, analysis of intraepidermal nerve fiber density and sciatic nerve morphology, respectively, as above, comparing between the three experimental groups and db/db untreated mice, between ages 16-24 weeks. We anticipate that the treatment will induce regeneration of the distal axons and recovery of myelinating and non-myelinating glia under diabetic conditions.

### Determine the Effect of Foretinib on Dermal and Corneal Wound Healing During Diabetes

In diabetics, DPN is associated with chronic wounds. We hypothesize that foretinib will prevent the impairments in topical wound healing in db/db mice. db/db mice develop severe and progressive wound healing deficits as early as 8 weeks of age. To test the effectiveness of foretinib, by maintaining SC and axonal health, improve wound healing, we will IP db/db mice with daily 5 mg/kg foretinib or vehicle for 12 weeks, starting at 4 weeks of age (n=8 per group per timepoint). Following 12 weeks of treatment, bilateral 6 mm full thickness back skin wounds will be created with a punch biopsy as done previously and wounds will be collected at 5-, 10-, and 15-days post injury. Foretinib treated non-diabetic db/+ (n=8 per group, per timepoint) mice will serve as controls. Twenty-four hours prior to sacrifice, the mice will be IP injected with EdU (20 mg/ml at 50 mg/kg) to track cell proliferation. Wounds will be excised, fixed in 4% PFA, cryopreserved, and mounted on slides. The extent of wound healing and dermal maturation will be assessed through histology and measurement of the wound width, wound area, dermal and epidermal thickness and collagen fiber assembly using standardized Masson's trichrome analysis. The rate of cell proliferation in the dermis and epidermis will be examined through the analysis of EdU and immunostaining for markers of dermal (PDGFRα) and epidermal cells (K5) in addition to assessing the extent of vascularization (CD31). As we demonstrated previously, under normal conditions after skin injury, SC dedifferentiate and infiltrate the wound bed area where they trophically stimulate wound healing. The total number of SC will be assessed through immunohistochemical staining for S100β and Sox10 while the proportion of dedifferentiated SC will be examined through co-staining for Sox2 and p75^{NTR}. Axon regeneration in healing skin will be quantified through immunohistochemical for GAP43 and βIII-tubulin (as above). Finally, using an animal model of Neurotrophic Keratopathy, we have previously shown that, similar to diabetic neuropathy in cornea, experimental corneal denervation impairs epithelial wound healing. As in skin, the nerve-associated Schwann cells regulate epithelial recovery in cornea. Here, we will assess the effect of foretinib treatment on corneal wound healing in db/db mice. To this end, we will remove the corneal epithelium in the experimental mice with a standard rotating (Amoils) brush and assess corneal healing over 4 days by fluorescein staining. We anticipate that foretinib will improve wound healing in both skin and cornea in db/db mice.

Further, poor wound healing worsens with long term hyperglycemia. Here, we will test the hypothesis that acute foretinib treatment during advanced DPN will accelerate wound repair. Using the same treatment groups/number of mice described above, we will perform the treatment for 8 weeks during advanced DPN (16-24 weeks of age). Mice will be wounded and skin collected at 5-, 10-, and 15-days post injury. The same analysis as above will be performed to compare wound healing outcomes, cell proliferation and vascularization in dependence of SC quantity and innervation extend across groups. We anticipate that foretinib will reverse diabetic impairment in wound healing, improve dermal cell proliferation and improve vascularization.

Sample size calculations are based on the previous report and preliminary studies on foretinib prevented diabetic neuropathy in mice. For the proposed splinted excisional wound experiments, n=8 based on power calculations; alpha=0.05, two-tail tests will be needed to achieve statistical significance.

Statistical interpretation and analysis of results: All samples will be blinded and data analyses will be performed by a statistician who is blinded to the groups. Data will be reported as mean±SD. For normally distributed data, Student's t-test or analysis of variance (ANOVA) will be applied. Multiple wounds in each animal will be handled using nested terms in the model. Non-parametric statistics will be applied if the data are not normally distributed. Significance level is set at 0.05. All analyses will be run by statistical core using Stata 13.1+, StataCorp, College Station, TX.

Currently, there are no effective treatments for DPN or impaired cutaneous wound healing in diabetic patients. Foretinib is the only safe-in-human compound with a strong neuroprotective activity, including in an animal model of DPN.

References, each of which are incorporated by reference herein:
1. Federation. ID. IDF Diabetes Atlas 9th edition. 2019 ed. Brussels, Belgium2019.
2. Maser RE, Steenkiste AR, Dorman JS, et al. Epidemiological correlates of diabetic neuropathy. Report from Pittsburgh Epidemiology of Diabetes Complications Study. Diabetes 1989;38:1456-61.
3. Ziegler D, Papanas N, Zhivov A, et al. Early detection of nerve fiber loss by corneal confocal microscopy and skin biopsy in recently diagnosed type 2 diabetes. Diabetes 2014;63:2454-63.
4. Leckelt J, Guimaraes P, Kott A, Ruggeri A, Stachs O, Baltrusch S. Early detection of diabetic neuropathy by investigating CNFL and IENFD in thy 1-YFP mice. The Journal of endocrinology 2016;231:147-57.
5. Abbott CA, Malik RA, van Ross ER, Kulkarni J, Boulton AJ. Prevalence and characteristics of painful diabetic neuropathy in a large community-based diabetic population in the U.K. Diabetes care 2011;34:2220-4.
6. Johannsen L, Smith T, Havsager AM, et al. Evaluation of patients with symptoms suggestive of chronic polyneuropathy. Journal of clinical neuromuscular disease 2001;3:47-52.
7 Dyck PJ, Kratz KM, Karnes JL, et al. The prevalence by staged severity of various types of diabetic neuropathy, retinopathy, and nephropathy in a population-based cohort: the Rochester Diabetic Neuropathy Study. Neurology 1993;43:817-24.
8. Zhang P, Lu J, Jing Y, Tang S, Zhu D, Bi Y. Global epidemiology of diabetic foot ulceration: a systematic review and meta-analysis (†). Ann Med 2017;49:106-16.
9 Schreml S, Berneburg M. The global burden of diabetic wounds. Br J Dermatol 2017;176:845-6.
10. Callaghan BC, Price RS, Feldman EL. Distal Symmetric Polyneuropathy: A Review. JAMA 2015;314:2172-81.
11. Scott JN, Clark AW, Zochodne DW. Neurofilament and tubulin gene expression in progressive experimental diabetes: failure of synthesis and export by sensory neurons. Brain 1999;122 (Pt 11):2109-18.
12. Goncalves NP, Vaegter CB, Andersen H, Ostergaard L, Calcutt NA, Jensen TS. Schwann cell interactions with axons and microvessels in diabetic neuropathy. Nature reviews Neurology 2017;13:135-47.
13. Gumy LF, Bampton ET, Tolkovsky AM. Hyperglycaemia inhibits Schwann cell proliferation and migration and restricts regeneration of axons and Schwann cells from adult murine DRG. Mol Cell Neurosci 2008;37:298-311.
14. Chowdhury SK, Smith DR, Fernyhough P. The role of aberrant mitochondrial bioenergetics in diabetic neuropathy. Neurobiology of disease 2013;51:56-65.
15. Sango K, Mizukami H, Horie H, Yagihashi S. Impaired Axonal Regeneration in Diabetes. Perspective on the Underlying Mechanism from In Vivo and In Vitro Experimental Studies. Frontiers in endocrinology 2017;8:12.
16. Wong MC, Chung JW, Wong TK. Effects of treatments for symptoms of painful diabetic neuropathy: systematic review. BMJ (Clinical research ed) 2007;335:87.
17. Feinberg K, Kolaj A, Wu C, et al. A neuroprotective agent that inactivates prodegenerative TrkA and preserves mitochondria. The Journal of cell biology 2017;216:3655-75.
18. Yau TCC, Lencioni R, Sukeepaisarnjaroen W, et al. A Phase I/II Multicenter Study of Single-Agent Foretinib as First-Line Therapy in Patients with Advanced Hepatocellular Carcinoma. Clinical cancer research: an official journal of the American Association for Cancer Research 2017;23:2405-13.
19. Seiwert T, Sarantopoulos J, Kallender H, McCallum S, Keer HN, Blumenschein G, Jr. Phase II trial of single-agent foretinib (GSK1363089) in patients with recurrent or metastatic squamous cell carcinoma of the head and neck. Investigational new drugs 2013;31:417-24.
20. Choueiri TK, Vaishampayan U, Rosenberg JE, et al. Phase II and biomarker study of the dual MET/VEGFR2 inhibitor foretinib in patients with papillary renal cell carcinoma. Journal of clinical oncology: official journal of the American Society of Clinical Oncology 2013;31:181-6.
21. Malik RA. Pathology of human diabetic neuropathy. Handb Clin Neurol 2014;126:249-59.
22. Chen HM, Tsai CH, Hung WC. Foretinib inhibits angiogenesis, lymphangiogenesis and tumor growth of pancreatic cancer in vivo by decreasing VEGFR-2/3 and TIE-2 signaling. Oncotarget 2015;6:14940-52.
23. Nørgaard SA, Sand FW, Sørensen DB, Abelson KS, Søndergaard H. Softened food reduces weight loss in the streptozotocin-induced male mouse model of diabetic nephropathy. Lab Anim 2018;52:373-83.
24. Biessels GJ, Bril V, Calcutt NA, et al. Phenotyping animal models of diabetic neuropathy: a consensus statement of the diabetic neuropathy study group of the EASD (Neurodiab). Journal of the peripheral nervous system: JPNS 2014;19:77-87.
25. Cai D, Zhu M, Petroll WM, Koppaka V, Robertson DM. The impact of type 1 diabetes mellitus on corneal epithelial nerve morphology and the corneal epithelium. Am J Pathol 2014; 184:2662-70.
26. Sullivan KA, Hayes JM, Wiggin TD, et al. Mouse models of diabetic neuropathy. Neurobiology of disease 2007;28:276-85.
27. Jolivalt CG, Frizzi KE, Guernsey L, et al. Phenotyping Peripheral Neuropathy in Mouse Models of Diabetes. Current protocols in mouse biology 2016;6:223-55.
28. Qi Y, Yu T, Xu J, et al. FDISCO: Advanced solvent-based clearing method for imaging whole organs. Sci Adv 2019;5:eaau8355.
29. Zaimi A, Wabartha M, Herman V, Antonsanti PL, Perone CS, Cohen-Adad J. AxonDeepSeg: automatic axon and myelin segmentation from microscopy data using convolutional neural networks. Scientific reports 2018;8:3816.
1 Srinivasan, E. & Rajasekaran, R. A Systematic and Comprehensive Review on Disease-Causing Genes in Amyotrophic Lateral Sclerosis. J Mol Neurosci 70, 1742-1770 (2020). doi.org:10.1007/s12031-020-01569-w
2 Ferraiuolo, L., Kirby, J., Grierson, A. J., Sendtner, M. & Shaw, P. J. Molecular pathways of motor neuron injury in amyotrophic lateral sclerosis. Nat Rev Neurol 7, 616-630 (2011). doi.org:10.1038/nrneurol.2011.152
3 Beard, J. D. & Kamel, F. Military service, deployments, and exposures in relation to amyotrophic lateral sclerosis etiology and survival. Epidemiol Rev 37, 55-70(2015). doi.org:10.1093/epirev/mxu001
4 Beard, J. D. et al. Military service, deployments, and exposures in relation to amyotrophic lateral sclerosis survival. PLOS One 12, e0185751 (2017). doi.org: 10.1371/journal.pone.0185751
5 Chio, A., Mazzini, L. & Mora, G. Disease-modifying therapies in amyotrophic lateral sclerosis. Neuropharmacology 167, 107986 (2020). doi.org:10.1016/j.neuropharm.2020.107986
6 Johnson, S. A. et al. Pharmacotherapy for Amyotrophic Lateral Sclerosis: A Review of Approved and Upcoming Agents. Drugs 82, 1367-1388 (2022). doi.org:10.1007/s40265-022-01769-1
7 Rosen, D. R. Mutations in Cu/Zn superoxide dismutase gene are associated with familial amyotrophic lateral sclerosis. Nature 364, 362 (1993). doi.org:10.1038/364362c0
8 Gurney, M. E. et al. Motor neuron degeneration in mice that express a human Cu,Zn superoxide dismutase mutation. Science 264, 1772-1775 (1994). doi.org:10.1126/science.8209258
9 Tu, P. H. et al. Transgenic mice carrying a human mutant superoxide dismutase transgene develop neuronal cytoskeletal pathology resembling human amyotrophic lateral sclerosis lesions. Proc Natl Acad Sci USA 93, 3155-3160 (1996). doi.org:10.1073/pnas.93.7.3155
10 Turner, B. J. & Talbot, K. Transgenics, toxicity and therapeutics in rodent models of mutant SOD1-mediated familial ALS. Prog Neurobiol 85, 94-134 (2008). doi.org: 10.1016/j.pneurobio.2008.01.001
11 Ticozzi, N. et al. Genetics of familial Amyotrophic lateral sclerosis. Arch Ital Biol 149, 65-82 (2011). doi.org:10.4449/aib.v14911.1262
12 Neumann, M. et al. Ubiquitinated TDP-43 in frontotemporal lobar degeneration and amyotrophic lateral sclerosis. Science 314, 130-133 (2006). doi.org:10.1126/science.1134108
13 Hong, K. et al. Full-length TDP-43 and its C-terminal fragments activate mitophagy in NSC34 cell line. Neurosci Lett 530, 144-149 (2012). doi.org:10.1016/j.neulet.2012.10.003
14 Magrane, J., Cortez, C., Gan, W. B. & Manfredi, G. Abnormal mitochondrial transport and morphology are common pathological denominators in SOD1 and TDP43 ALS mouse models. Hum Mol Genet 23, 1413-1424 (2014). doi.org:10.1093/hmg/ddt528
15 Wang, W. et al. The ALS disease-associated mutant TDP-43 impairs mitochondrial dynamics and function in motor neurons. Hum Mol Genet 22, 4706-4719 (2013). doi.org:10.1093/hmg/ddt319
16 Atsumi, T. The ultrastructure of intramuscular nerves in amyotrophic lateral sclerosis. Acta Neuropathol 55, 193-198 (1981). doi.org:10.1007/BF00691318
17 Sasaki, S. & Iwata, M. Mitochondrial alterations in the spinal cord of patients with sporadic amyotrophic lateral sclerosis. J Neuropathol Exp Neurol 66, 10-16 (2007). doi.org:10.1097/nen.0b013e31802c396b
18 Takahashi, K., Nakamura, H. & Okada, E. Hereditary amyotrophic lateral sclerosis. Histochemical and electron microscopic study of hyaline inclusions in motor neurons. Arch Neurol 27, 292-299 (1972). doi.org:10.1001/archneur. 1972.00490160020003
19 Sun, C. N., Araoz, C., Lucas, G., Morgan, P. N. & White, H. J. Amyotrophic lateral sclerosis. Inclusion bodies in a case of the classic sporadic form. Ann Clin Lab Sci 5, 38-44 (1975).
20 Warita, H., Itoyama, Y. & Abe, K. Selective impairment of fast anterograde axonal transport in the peripheral nerves of asymptomatic transgenic mice with a G93A mutant SODI gene. Brain Res 819, 120-131 (1999). doi.org:10.1016/s0006-8993 (98) 01351-1
21 Williamson, T. L. & Cleveland, D. W. Slowing of axonal transport is a very early event in the toxicity of ALS-linked SOD1 mutants to motor neurons. Nat Neurosci 2, 50-56 (1999). doi.org:10.1038/4553
22 Shi, P., Gal, J., Kwinter, D. M., Liu, X. & Zhu, H. Mitochondrial dysfunction in amyotrophic lateral sclerosis. Biochim Biophys Acta 1802, 45-51 (2010). doi.org:10.1016/j.bbadis.2009.08.012
23 Philips, T. & Robberecht, W. Neuroinflammation in amyotrophic lateral sclerosis: role of glial activation in motor neuron disease. Lancet Neurol 10, 253-263 (2011). doi.org:10.1016/S1474-4422(11)70015-1
24 Blokhuis, A. M., Groen, E. J., Koppers, M., van den Berg, L. H. & Pasterkamp, R. J. Protein aggregation in amyotrophic lateral sclerosis. Acta Neuropathol 125, 777-794 (2013). doi.org:10.1007/s00401-013-1125-6
25 Zhang, K. et al. The C9orf72 repeat expansion disrupts nucleocytoplasmic transport. Nature 525, 56-61 (2015). doi.org:10.1038/nature14973
26 Wosiski-Kuhn, M., Lyon, M. S., Caress, J. & Milligan, C. Inflammation, immunity, and amyotrophic lateral sclerosis: II. immune-modulating therapies. Muscle Nerve 59, 23-33 (2019). doi.org:10.1002/mus.26288
27 Gitcho, M. A. et al. TDP-43 A315T mutation in familial motor neuron disease. Ann Neurol 63, 535-538 (2008). doi.org:10.1002/ana.21344
28 Sreedharan, J. et al. TDP-43 mutations in familial and sporadic amyotrophic lateral sclerosis. Science 319, 1668-1672 (2008). doi.org:10.1126/science.1154584
29 Kwiatkowski, T. J., Jr. et al. Mutations in the FUS/TLS gene on chromosome 16 cause familial amyotrophic lateral sclerosis. Science 323, 1205-1208 (2009). doi.org:10.1126/science.1166066
30 Deng, J. et al. FUS Interacts with HSP60 to Promote Mitochondrial Damage. PLOS Genet 11, e1005357 (2015). doi.org:10.1371/journal.pgen. 1005357
31 Higgins, C. M., Jung, C., Ding, H. & Xu, Z. Mutant Cu, Zn superoxide dismutase that causes motoneuron degeneration is present in mitochondria in the CNS. J Neurosci 22, RC215 (2002). doi.org:10.1523/JNEUROSCI.22-06-j0001.2002
32 Wang, W. et al. The inhibition of TDP-43 mitochondrial localization blocks its neuronal toxicity. Nat Med 22, 869-878 (2016). doi.org:10.1038/nm.4130
33 Lopez-Gonzalez, R. et al. Poly (GR) in C9ORF72-Related ALS/FTD Compromises Mitochondrial Function and Increases Oxidative Stress and DNA Damage in iPSC-Derived Motor Neurons. Neuron 92, 383-391 (2016). doi.org:10.1016/j.neuron.2016.09.015
34 Blokhuis, A. M. et al. Comparative interactomics analysis of different ALS-associated proteins identifies converging molecular pathways. Acta Neuropathol 132, 175-196(2016). doi.org:10.1007/s00401-016-1575-8
35 Mattiazzi, M. et al. Mutated human SODI causes dysfunction of oxidative phosphorylation in mitochondria of transgenic mice. J Biol Chem 277, 29626-29633 (2002). doi.org:10.1074/jbc.M203065200
36 Wiedemann, F. R., Manfredi, G., Mawrin, C., Beal, M. F. & Schon, E. A. Mitochondrial DNA and respiratory chain function in spinal cords of ALS patients. J Neurochem 80, 616-625 (2002). doi.org:10.1046/j.0022-3042.2001.00731.x
37 Borthwick, G. M., Johnson, M. A., Ince, P. G., Shaw, P. J. & Turnbull, D. M. Mitochondrial enzyme activity in amyotrophic lateral sclerosis: implications for the role of mitochondria in neuronal cell death. Ann Neurol 46, 787-790 (1999). doi.org:10.1002/1531-8249(199911)46:5<787::aid-ana17>3.0.co;2-8
38 Wiedemann, F. R. et al. Impairment of mitochondrial function in skeletal muscle of patients with amyotrophic lateral sclerosis. J Neurol Sci 156, 65-72 (1998). doi.org:10.1016/s0022-510x(98)00008-2
39 Vielhaber, S. et al. Mitochondrial DNA abnormalities in skeletal muscle of patients with sporadic amyotrophic lateral sclerosis. Brain 123 (Pt 7), 1339-1348 (2000). doi.org:10.1093/brain/123.7.1339
40 Crugnola, V. et al. Mitochondrial respiratory chain dysfunction in muscle from patients with amyotrophic lateral sclerosis. Arch Neurol 67, 849-854 (2010). doi.org:10.1001/archneurol.2010.128
41 Shah, M. A. et al. Phase II study evaluating 2 dosing schedules of oral foretinib (GSK1363089), cMET/VEGFR2 inhibitor, in patients with metastatic gastric cancer. PLOS One 8, e54014 (2013). doi.org:10.1371/journal.pone.0054014
42 Yau, T. C. C. et al. A Phase I/II Multicenter Study of Single-Agent Foretinib as First-Line Therapy in Patients with Advanced Hepatocellular Carcinoma. Clin Cancer Res 23, 2405-2413 (2017). doi.org: 10.1158/1078-0432.CCR-16-1789
43 Feinberg, K. et al. A neuroprotective agent that inactivates prodegenerative TrkA and preserves mitochondria. J Cell Biol 216, 3655-3675 (2017). doi.org:10.1083/jcb.201705085
44 Daeschler, S. C., Zhang, J., Gordon, T., Borschel, G. H. & Feinberg, K. Foretinib mitigates cutaneous nerve fiber loss in experimental diabetic neuropathy. Sci Rep 12, 8444 (2022). doi.org:10.1038/s41598-022-12455-3
45 Chowdhury, S. K., Smith, D. R. & Fernyhough, P. The role of aberrant mitochondrial bioenergetics in diabetic neuropathy. Neurobiol Dis 51, 56-65 (2013). doi.org:10.1016/j.nbd.2012.03.016
46 Bose, P., Armstrong, G. A. B. & Drapeau, P. Neuromuscular junction abnormalities in a zebrafish loss-of-function model of TDP-43. J Neurophysiol 121, 285-297 (2019). doi.org:10.1152/jn.00265.2018
47 Shadrach, J. L. et al. Translatomic analysis of regenerating and degenerating spinal motor neurons in injury and ALS. iScience 24, 102700 (2021). doi.org:10.1016/j.isci.2021.102700
48 Chen, H. M., Tsai, C. H. & Hung, W. C. Foretinib inhibits angiogenesis, lymphangiogenesis and tumor growth of pancreatic cancer in vivo by decreasing VEGFR-2/3 and TIE-2 signaling. Oncotarget 6, 14940-14952 (2015). doi.org:10.18632/oncotarget.3613
49 Smith, E. F., Shaw, P. J. & De Vos, K. J. The role of mitochondria in amyotrophic lateral sclerosis.Neurosci Lett 710, 132933 (2019). doi.org:10.1016/j.neulet.2017.06.052
50 Alves, C. J. et al. Early motor and electrophysiological changes in transgenic mouse model of amyotrophic lateral sclerosis and gender differences on clinical outcome. Brain Res 1394, 90-104 (2011). doi.org:10.1016/j.brainres.2011.02.060
51 Olivan, S. et al. Comparative study of behavioural tests in the SOD1G93A mouse model of amyotrophic lateral sclerosis. Exp Anim 64, 147-153 (2015). doi.org:10.1538/expanim.14-0077
52 Fischer, L. R. et al. Amyotrophic lateral sclerosis is a distal axonopathy: evidence in mice and man. Exp Neurol 185, 232-240 (2004). doi.org:10.1016/j.expneurol.2003.10.004
53 Smittkamp, S. E., Brown, J. W. & Stanford, J. A. Time-course and characterization of orolingual motor deficits in B6SJL-Tg(SOD1-G93A)1Gur/J mice. Neuroscience 151, 613-621 (2008). doi.org:10.1016/j.neuroscience.2007.10.017
54 Weydt, P., Hong, S. Y., Kliot, M. & Moller, T. Assessing disease onset and progression in the SODI mouse model of ALS. Neuroreport 14, 1051-1054 (2003). doi.org:10.1097/01.wnr.0000073685.00308.89
55 Wu, C., Watts, M. E. & Rubin, L. L. MAP4K4 Activation Mediates Motor Neuron Degeneration in Amyotrophic Lateral Sclerosis. Cell Rep 26, 1143-1156 e1145 (2019). doi.org:10.1016/j.celrep.2019.01.019
56 Tateishi, T. et al. CSF chemokine alterations related to the clinical course of amyotrophic lateral sclerosis. J Neuroimmunol 222, 76-81 (2010). doi.org:10.1016/j.jneuroim.2010.03.004
57 Gupta, P. K., Prabhakar, S., Sharma, S. & Anand, A. A predictive model for amyotrophic lateral sclerosis (ALS) diagnosis. J Neurol Sci 312, 68-72 (2012). doi.org:10.1016/j.jns.2011.08.021
58 Kawaguchi-Niida, M., Yamamoto, T., Kato, Y., Inose, Y. & Shibata, N. MCP-1/CCR2 signaling-mediated astrocytosis is accelerated in a transgenic mouse model of SOD1-mutated familial ALS. Acta Neuropathol Commun 1, 21 (2013). doi.org:10.1186/2051-5960-1-21
59 Nardo, G. et al. Immune response in peripheral axons delays disease progression in SODI (G93A) mice. J Neuroinflammation 13, 261 (2016). doi.org:10.1186/s12974-016-0732-2
60 Fiala, M., Mizwicki, M. T., Weitzman, R., Magpantay, L. & Nishimoto, N. Tocilizumab infusion therapy normalizes inflammation in sporadic ALS patients. Am J Neurodegener Dis 2, 129-139 (2013).
61 Schito, P. et al. Clinical Features and Biomarkers to Differentiate Primary and Amyotrophic Lateral Sclerosis in Patients With an Upper Motor Neuron Syndrome. Neurology (2023). doi.org:10.1212/WNL.0000000000207223
62 Pagliuca, F. W. et al. Generation of functional human pancreatic beta cells in vitro. Cell 159, 428-439 (2014). doi.org:10.1016/j.cell.2014.09.040
63 Maury, Y. et al. Combinatorial analysis of developmental cues efficiently converts human pluripotent stem cells into multiple neuronal subtypes. Nat Biotechnol 33, 89-96 (2015). doi.org:10.1038/nbt.3049
64 Bose, P. et al. Correction to: The Novel Small Molecule TRVA242 Stabilizes Neuromuscular Junction Defects in Multiple Animal Models of Amyotrophic Lateral Sclerosis. Neurotherapeutics 18, 2128 (2021). doi.org:10.1007/s13311-021-01061-2

1. A method of treating neurodegeneration in a subject in need thereof comprising administering a therapeutically effective amount of foretinib to the subject to treat the neurodegeneration.

2. The method of claim 1, wherein the subject has been diagnosed with amyotrophic lateral sclerosis (ALS).

3. The method of claim 2, wherein the subject has been diagnosed with end-stage ALS.

4. The method of claim 2, wherein the method induces the growth of new neuromuscular junctions in the subject.

5. The method of claim 3, wherein the method improves respiration or swallowing in the subject.

6. The method of claim 1, wherein the subject has been diagnosed with Alzheimer's disease.

7. The method of claim 1, wherein the subject has been diagnosed with diabetes.

8. The method of claim 7, wherein the subject has been diagnosed with diabetic neuropathy.

9. The method of claim 7, wherein the method reduces loss of epidermal nerve fiber density compared to a subject that has not been administered foretinib.

10. The method of claim 9, wherein the method does not reduce blood glucose levels in the subject.

11. The method of claim 1, wherein the method increases the axonal length of at least one neuron in the subject.

12. The method of claim 1, wherein administering comprises oral or intravenous administration.

13. The method of claim 1, wherein administering comprises intradermal or subcutaneous administration.

14. A method of restoring motor control in a subject with amyotrophic lateral sclerosis (ALS), the method comprising administering a therapeutically effective amount of foretinib to the subject to restore motor control in the subject.

15. The method of claim 14, wherein the subject has been diagnosed with end-stage ALS.

16. The method of claim 14, wherein the method induces the growth of new neuromuscular junctions in the subject.

17. The method of claim 14, wherein the method improves respiration or swallowing in the subject.

18. A method of reducing myelin loss in peripheral nerves in a subject suffering from multiple sclerosis, the method comprising administering a therapeutically effective amount of foretinib to the subject to reduce myelin loss in peripheral nerves.

19. The method of claim 18, wherein administering comprises oral or intravenous administration.

20.-33. (canceled)
